(19) United States
(12) Patent Application Publication
(10) Pub. No.: US 2026/0256483 A1
(43) Pub. Date: Sep. 3, 2026

(54) LITHOTRIPSY BALLOON DILATION CATHETERS HAVING BALLOONS TO CONFORM TO LESIONS THAT ARE TARGETED BY AN ACOUSTIC OR SOUND WAVE

(71) Applicant: Cardiovascular Systems, Inc., Santa Clara, CA (US)

(72) Inventors: Michael J. Morris, Lismore (IE); Kathy A. Kaveney, Carlsbad, CA (US); Emmet M. Lonergan, Co Tipperary (IE); Diarmuid J. Wall, Co Tipperary (IE); Jonathan P. Durcan, Temecula, CA (US)

(21) Appl. No.: 19/551,233

(22) Filed: Feb. 26, 2026

Related U.S. Application Data
(60) Provisional application No. 63/764,913, filed on Feb. 28, 2025.

(51) Int. Cl. A61B 17/22; A61B 17/00; A61B 18/00; A61B 18/24; A61B 18/26; A61M 25/10

(52) U.S. Cl. CPC A61B 17/22022; A61B 17/00234; A61B 18/245; A61B 18/26; A61M 25/10; A61B 2017/00238; A61B 2017/00305; A61B 2017/00544; A61B 2017/22025; A61B 2017/22038; A61B 2017/22065; A61B 2017/22098; A61B 2018/00369; A61B 2018/263; A61M 2025/1075; A61M 2025/1084; A61M 2025/1088; A61M 2025/1093; A61M 2205/0222; A61M 2210/125

(57) ABSTRACT

Methods and devices for lithotripsy procedures. A lithotripsy balloon catheter includes an elongate catheter shaft, an inflation lumen, a multilayer inflatable balloon in fluid communication with the inflation lumen located toward, or at, a distal section of the elongate catheter shaft, and at least one lithotripsy emitter within the multilayer inflatable balloon capable of generating an acoustic pressure wave within an inflation medium when the multilayer inflatable balloon is inflated. The multilayer inflatable balloon includes a first layer and a second layer having a combined wall thickness. The first layer is made of a first polymer material and the second layer is made of a second polymer material, where the second layer is an inner layer relative to the first layer. The multilayer inflatable balloons in accordance with the present disclosure may exhibit a higher rupture strength as compared to a similar, but monolithic, balloon.

### CROSS-REFERENCE TO RELATED APPLICATION

The present application claims the benefit of U.S. Patent Application Ser. No. 63/764,913 filed on Feb. 28, 2025, and entitled “LITHOTRIPSY BALLOON DILATION CATHETERS HAVING BALLOONS TO CONFORM TO LESIONS THAT ARE TARGETED BY SOUND WAVE,” which application is expressly incorporated herein by reference in its entirety.

### BACKGROUND OF THE INVENTION

### Technical Field

The present disclosure pertains generally to the field of intravascular and other medical devices for insertion into the vasculature or other bodily vessels, and more particularly to such devices that include a balloon for use in lithotripsy procedures within such vessels. Intravascular lithotripsy (IVL) is an example of such, although additional examples include use of a lithotripsy balloon catheter within a heart valve lumen or heart valve leaflet vessel lumen. While IVL may be exemplary, it will be appreciated that the present devices and methods extend to other such uses.

### Description of the Related Art

Calcification of the vasculature and other vessels refers to the accumulation of calcium deposits on or within the walls of such vessels, which can significantly reduce blood flow and increase the risk of cardiovascular events like heart attacks and strokes, as well as conditions such as chronic kidney disease and diabetes. As such, it is a serious medical concern. Additionally, vascular calcification reduces arterial compliance and complicates both short- and long-term clinical outcomes following revascularization treatments. Coronary artery calcification is reported in 18% to 31% of percutaneous procedures, while the incidence in peripheral arterial procedures is 30% to 50%.

Coronary artery calcification negatively impacts the outcomes of coronary interventions by hindering device passage, as well as stent placement and expansion, and by causing delamination of drug-eluting polymers, which disrupts drug delivery and elution. Likewise, peripheral artery calcification limits vessel expansion, leading to greater residual stenosis, which decreases the effectiveness of procedures and may weaken the antiproliferative effect of drug-coated balloons.

Methods for treating coronary artery calcification, such as use of noncompliant, cutting, and scoring balloons, as well as atherectomy (e.g., atheroablative) technologies, have their own limitations. High-pressure balloon dilation with noncompliant or specialty balloons may not provide enough force to fracture calcium and expand the vessel, potentially causing barotrauma-related dissection or perforation. Laser atherectomy can be unpredictable. Peri-procedural complications, including slow flow, myocardial infarction, flow-limiting dissection, distal embolization, and perforation, are more common when atheroablative technologies are used in conjunction with balloons, compared to using balloons alone.

Likewise with peripheral artery calcification, endovascular treatment with percutaneous transluminal angioplasty or stenting is associated with higher rates of dissection, perforation, and distal embolization, along with lower long-term patency when peripheral artery calcification is present.

Intravascular lithotripsy (IVL) has emerged as a treatment for vascular calcification. In IVL, the shaft of a balloon angioplasty catheter contains lithotripsy emitters (which generate acoustic pressure waves), allowing localized wave pulses to be delivered circumferentially for the disruption of vascular calcium. In this way, IVL converts electrical energy into mechanical energy by creating sonic pressure waves (e.g., generated by vaporizing the fluid within the balloon) that selectively disrupts calcium deposits. The sonic pressure waves created may exert up to approximately 55 atm of pressure outside of the balloon, which is transferred to the vessel, and lesion, thereby breaking both superficial and deep calcium deposits within the vessel wall. In this way, IVL procedures increase vessel compliance by creating microfractures in the calcified plaque. Vessel compliance is important for adequate balloon and stent expansion, in addition to general coronary and vascular health. Importantly, the sonic pressure waves selectively disrupt calcified plaques, while causing no significant harm to non-calcified, healthy soft tissue. In contrast to calcified tissue, sonic pressure waves have a high transmission through (rather than reflection from) non-calcified tissue, e.g., because there are minimal differences in acoustic impedance between the medium carrying the sonic waves and the soft tissue, which is composed primarily of water. The safety and effectiveness of IVL have been demonstrated in several clinical studies involving severely calcified coronary and peripheral artery disease.

One continuing problem with existing lithotripsy balloon catheters is that conventional balloons employed for IVL and other lithotripsy procedures too often suffer from balloon failure, e.g., due to premature rupture during use. Such balloon failure is unacceptable, as it can lead to potential complications such as vessel dissection, vessel rupture or embolization.

Various efforts have been made to increase balloon integrity in an IVL system. See, for example, U.S. Pat. Application Publication No. 2021/0378743 that describes various balloon integrity protection components.

Other efforts have focused on increasing the wall thickness of conventional lithotripsy balloons, although this is problematic as it increases the size of the overall catheter system to be inserted into the vasculature or other body lumens, where space is already at a premium.

Therefore, while balloon catheters for lithotripsy exist, there continues to be a need in the art for improved lithotripsy balloon catheters, particularly with improved rupture resistance for such balloons. It would be a further advantage if such could be achieved using similar balloon materials, but without any significant increase in balloon thickness.

### SUMMARY

This Summary is provided to introduce a selection of concepts in a simplified form that are further described below in the Detailed Description. This Summary is not intended to identify key features or essential features of the claimed subject matter. Embodiments of the present disclosure provide systems, methods, and devices for a multilayer balloon catheter for use in lithotripsy procedures within a patient's body.

An exemplary lithotripsy balloon catheter system may typically include: (1) a generator (e.g., portable and/or rechargeable) that generates electric pulses; (2) a connector cable that connects the generator to a control, e.g., with a push button, allowing delivery of electronic pulses and, thereby, the formation of sonic or acoustic pressure waves; and (3) a balloon catheter, e.g. a compliant or semi-compliant balloon catheter, equipped with an electrode pair or other lithotripsy emitter (e.g., one or more lasers and associated target(s)). The balloon may include radiopaque markers at the ends of the balloon, to allow the practitioner to visualize and confirm correct positioning. The balloon is configured to be filled with a fluid medium, such as a mixture of 0.9% NaCl saline solution and contrast media (e.g., 50% by volume or weight of each). During a lithotripsy procedure, the balloon may be at least partially inflated, to an inflation pressure significantly below that typically used for balloon angioplasty (e.g., only about 4 atm), sufficient to provide firm apposition of the balloon against the calcified vessel, but insufficient to provide significant pressure to such vessel. A number of lithotripsy pulses may be delivered. After completion of such a cycle, the inflation pressure may be increased (e.g., to about 6 atm or more) before the lithotripsy balloon is deflated.

A rated burst pressure of a conventional IVL balloon may only be about 10 atm. The presently described systems may include balloons rated to a higher burst pressure with substantially identical thickness, or such balloons may be thinner, but rated to a substantially similar burst pressure.

During a given lithotripsy procedure, up to about 120 pulses, or up to about 80 pulses may be provided, e.g., at a frequency of about 1 Hz.

An exemplary lithotripsy procedure may be as follows:
1. Place lithotripsy balloon catheter in vasculature containing a calcified plaque.
2. Once the lithotripsy balloon catheter is in place, record position using fluoroscopy.
3. If position is incorrect, adjust the lithotripsy balloon to the correct position.
4. Inflate the lithotripsy balloon to 4.0 atm.
5. Deliver pressure wave sequence. Pressure wave sequence may have the following typical parameters: treatment frequency of 1 pulse per second; the maximum number of continuous pulses in 1 cycle may be 20 pulses; the minimum pause time may be 10 seconds.
6. Inflate balloon to a degree that takes into account the balloon's compliance and record lesion response on fluoroscopy.
7. Following lithotripsy treatment, deflate balloon. A wait time may then be observed, for example 30 seconds, to re-establish blood flow.
8. Repeat steps 3, 4, 5, 6 to complete a treatment, for example with a total of 40 pulses.
9. Additional treatments can be performed if necessary. If multiple inflations are required due to a lesion length greater than the lithotripsy balloon length, the recommended balloon overlap between inflation locations is at least 1 cm to prevent geographic miss. Care should be taken to not exceed a predetermined number of pulses, such as 160 pulses, in the same segment, and it may be necessary to remove and replace the balloon catheter, if more than a threshold maximum number of pulses have been applied (e.g., 160 pulses) using a given balloon.

An embodiment of the present disclosure is directed to a balloon catheter for use in a lithotripsy procedure ((IVL or otherwise), comprising an elongate catheter shaft, an inflation lumen, a multilayer inflatable balloon in fluid communication with the inflation lumen located toward, or at, a distal section of the elongate catheter shaft, and at least one lithotripsy emitter (e.g., an electrode pair, laser or other lithotripsy emitter) within the multilayer inflatable balloon capable of generating a pressure wave within an inflation medium when the multilayer inflatable balloon is inflated. The multilayer inflatable balloon includes a first layer and a second layer (an inner layer relative to the first layer) having a combined wall thickness, where the first layer comprises a first polymer material and the second layer comprises a second polymer material. The first and second layers provide a rupture strength that is substantially equal to or greater than that of a comparative single layer balloon made entirely of the first polymer material or entirely of the second polymer material, with a wall thickness equal or substantially equal to the combined wall thickness. In this comparison of the two devices, other characteristics and/or configurations of the devices are unchanged. In an embodiment, the first polymer material of the first layer has a first maximum blow-up-ratio, and the second polymer material of the second layer has a second maximum blow-up-ratio that is greater than the first maximum blow-up-ratio. By way of example, positioning the higher blow-up ratio material as the second layer which is an inner layer relative to the first layer, achieves such an increase in rupture strength. The higher blow-up ratio material of the inner layer may be softer (i.e., lower durometer) than the first layer. The hardness of the materials may be measured using a durometer, according to the Shore D hardness scale, for example following the standard set out in ASTM D2240.

Another embodiment of the present disclosure is directed to a balloon catheter for use in a lithotripsy procedure, comprising an elongate catheter shaft, an inflation lumen, a multilayer inflatable balloon in fluid communication with the inflation lumen located toward, or at, a distal section of the elongate catheter shaft, and at least one lithotripsy emitter (e.g., an electrode pair, laser or other lithotripsy emitter) within the multilayer inflatable balloon capable of generating a pressure wave within an inflation medium when the multilayer inflatable balloon is inflated. The multilayer inflatable balloon includes a first layer and a second layer (an inner layer relative to the first layer) having a combined wall thickness, where the first layer is made of a first polymer material having a first maximum blow-up-ratio, and the second layer is made of a second polymer material having a second maximum blow-up-ratio greater than the first maximum blow-up-ratio.

Another embodiment of the present disclosure is directed to a method of manufacturing a balloon catheter. Such a method may comprise providing a balloon catheter comprising an elongate catheter shaft and an inflation lumen, a multilayer inflatable balloon in fluid communication with the inflation lumen located toward, or at, a distal section of the elongate catheter shaft, and at least one lithotripsy emitter (e.g., an electrode pair, laser or other lithotripsy emitter) within the multilayer inflatable balloon capable of generating a pressure wave within an inflation medium when the multilayer inflatable balloon is inflated. The multilayer inflatable balloon comprises a first layer and a second layer (an inner layer relative to the first layer) having a combined wall thickness. The first layer is made of a first polymer material and the second layer is made of a second polymer material. The blow-up ratio of the second polymer material may be greater than that of the first polymer material. The method further includes folding the multilayer layer balloon along one or more pleat lines, where the multilayer inflatable balloon is positioned about the elongate catheter shaft, compressing the folded multilayer inflatable balloon using a balloon press, removing the balloon press from about the multilayer inflatable balloon, and positioning a balloon sheath over the compressed balloon to maintain a compressed geometry of the multilayer inflatable balloon.

Another embodiment of the present disclosure is directed to a method for using a balloon catheter in a lithotripsy procedure (IVL or otherwise). Such a method may include providing a lithotripsy balloon catheter, inserting the lithotripsy balloon catheter into a vessel lumen to be treated, and inflating the lithotripsy balloon using the inflation medium, to a relatively low inflation pressure of no greater than about 5 atm, so as to position the inflated multilayer inflatable balloon against an interior of a wall of the vessel lumen, in which the inflation pressure is sufficiently low so as to provide apposition against the vessel wall, but not to exert significant pressure against the vessel wall, and applying power to at least one lithotripsy emitter (e.g., an electrode pair, laser or other lithotripsy emitter) of the balloon catheter, so as to generate a pressure wave within the multilayer inflatable balloon, the pressure wave propagating through the inflation medium and through the thickness of the multilayer inflatable balloon and into the wall of the vessel lumen, so as to break up calcified tissue associated with the vessel wall. The balloon catheter may include an elongate catheter shaft having a proximal section and a distal section, an inflation lumen, a multilayer inflatable balloon located toward, or at, a distal section of the elongate catheter shaft, and at least one lithotripsy emitter (e.g., an electrode pair, laser or other lithotripsy emitter) within the multilayer inflatable balloon capable of generating a pressure wave within an inflation medium when the multilayer inflatable balloon is inflated during use. The multilayer inflatable balloon includes at least a first layer and a second layer (an inner layer relative to the first layer) having a combined wall thickness, where the first layer is made of a first polymer material and the second layer is made of a second polymer material. The first polymer material of the first may have a first maximum blow-up-ratio, and the second material of the second layer may have a second maximum blow-up-ratio that is greater than the first maximum blow-up-ratio.

Another embodiment of the present disclosure is directed to a method for using a balloon catheter in a heart valve lithotripsy procedure or in a heart valve leaflet lithotripsy procedure, the method comprising providing a lithotripsy balloon catheter, inserting the lithotripsy balloon catheter into a heart valve vessel lumen or heart valve leaflet vessel lumen to be treated, and inflating the lithotripsy balloon using the inflation medium, to a relatively low inflation pressure of no greater than about 5 atm, so as to position the inflated multilayer inflatable balloon against an interior of a wall of the vessel lumen, in which the inflation pressure is sufficiently low so as to provide apposition against the vessel wall, but not to exert significant pressure against the vessel wall, and applying power to at least one lithotripsy emitter (e.g., an electrode pair, laser or other lithotripsy emitter) of the lithotripsy balloon catheter so as to generate a pressure wave within the multilayer inflatable balloon, the pressure wave propagating through the inflation medium and through the thickness of the multilayer inflatable balloon and into the wall of the vessel lumen, so as to break up calcified tissue associated with the vessel wall. The balloon catheter may include an elongate catheter shaft having a proximal section and a distal section, an inflation lumen, a multilayer inflatable balloon located toward, or at, a distal section of the elongate catheter shaft, and the at least one lithotripsy emitter within the multilayer inflatable balloon capable of generating a pressure wave within the inflation medium when the multilayer inflatable balloon is inflated during use. The multilayer inflatable balloon includes at least a first layer and a second layer (an inner layer relative to the first layer) having a combined wall thickness, where the first layer is made of a first polymer material and the second layer is made of a second polymer material. The first polymer material may have a first maximum blow-up-ratio and the second polymer material may have a second maximum blow-up-ratio that is greater than the first maximum blow-up-ratio.

In any of the described embodiments, the balloon catheter may be such that the at least one lithotripsy emitter comprises at least one electrode pair. The balloon catheter may be configured to provide sufficient power to the electrodes within the multilayer inflatable balloon to generate a plasma arc within the inflation medium used to inflate the multilayer inflatable balloon. Generation of the plasma arc may advantageously result in a pressure wave within the multilayer inflatable balloon during the IVL procedure.

In any of the described embodiments, the balloon catheter may be such that the at least one lithotripsy emitter comprises one or more laser emitters. The balloon catheter may be configured to provide sufficient power to the laser emitters within the multilayer inflatable balloon to generate a pressure wave within the inflation medium used to inflate the multilayer inflatable balloon during the IVL procedure.

In any of the described embodiments, the first and second layers may provide a rupture strength that is substantially equal to or greater than that of a comparative single layer balloon made of the first polymer material with a wall thickness equal to the combined wall thickness. By way of example, rupture strength may be increased by at least 5%, at least 10%, at least 15%, or at least 20%.

In any of the described embodiments, the first and second layers may provide a rupture strength that is substantially equal to or greater than that of a comparative single layer balloon made of the second polymer material with a wall thickness equal to the combined wall thickness. By way of example, rupture strength may be increased by at least 5%, at least 10%, at least 15%, or at least 20%.

The multilayer balloon catheter of the present disclosure may be configured such that any reflection associated with the interface between the inner layer and the outer layer (or an interface between any additional layers) is minimized, or of low significance. Measurement, calculation or other determination of transmission and/or reflection characteristics and values may be difficult, or may be accomplished in various ways. For example, it may not be practical to directly measure the transmission between individual balloon layers. However if transmission of the wave from the lithotripsy emitter or inflation medium (e.g., measuring the generated wave) to external of the balloon were measured such measurement could be used to inform the transmission through the balloon and associated material layers. For example, transmission through the multilayer balloon can be compared to transmission through a monolithic single layer balloon, showing that transmission is not significantly reduced, or is perhaps even improved. For example, when making such a comparison, measured pressure may not be attenuated in the multilayer balloon as compared to a single layer balloon by any more than about 25%, about 20%, about 15%, about 10%, about 5%, or about 3%.

In any of the described embodiments, the density of the second layer may be within ±25%, ±20%, ±15%, ±10%, or ±5% of a density of the inner layer.

In any of the described embodiments, an inner portion of the inner layer may exhibit a higher degree of polymer chain orientation as compared to an outer portion of the inner layer. Chain orientation may be measured by any suitable technique.

In any of the described embodiments, an inner portion of the inner layer may exhibit a crystallinity that is within a range of about 30% to 100%, from about 35% to about 90%, or from about 40% to about 80%, and the outer portion of the inner layer exhibits a crystallinity that is within a range of 0% to about 30%, from about 0% to about 25%, or from about 0% to about 20%. Crystallinity may be measured by any suitable technique.

In any of the described embodiments, the first layer may be an outer layer, wherein an inner portion of the outer layer may exhibit a higher degree of polymer chain orientation as compared to an outer portion of the outer layer.

In any of the described embodiments, the inner portion of the outer layer may exhibit a crystallinity that is within a range of about 30% to 100%, from about 35% to about 90%, or from about 40% to about 80%, and the outer portion of the outer layer exhibits a crystallinity that is within a range of 0% to about 30%, from about 0% to about 25%, or from about 0% to about 20%.

In any of the described embodiments, the first layer may be an outer layer, wherein an inner portion of the inner layer may exhibit a higher degree of polymer chain orientation as compared to an outer portion of the inner layer, and an inner portion of the outer layer may exhibit a higher degree of polymer chain orientation as compared to an outer portion of the outer layer.

In any of the described embodiments, the inner layer may account for about 10% to about 40% of the combined wall thickness of the multilayer inflatable balloon.

In any of the described embodiments, the first layer may be an outer layer, wherein the outer layer accounts for about 60% to about 90% of the combined wall thickness of the multilayer inflatable balloon.

In any of the described embodiments, the multilayer inflatable balloon may include a total of from 2 to 5, from 2 to 4, or 2 or 3 layers.

In any of the described embodiments, the multilayer inflatable balloon may include only two layers.

In any of the described embodiments, the at least one lithotripsy emitter may include at least one electrode pair, or at least two electrode pairs (i.e., 4 electrodes).

In any of the described embodiments, the multilayer inflatable balloon may include a taper at a distal end of the balloon that differs from a taper at a proximal end of the balloon.

In any of the described embodiments, the taper at the distal end of the balloon may provide a more gradual transition in radial width when crossing a lesion, as compared to the taper at the proximal end of the balloon.

In any of the described embodiments, the taper at the distal end of the balloon may include a taper length of from about 3 mm to about 10 mm, and/or a taper angle of from 10° to about 25°.

In any of the described embodiments, the distal end of the balloon may provide less crossing resistance when crossing a lesion due to increased taper length and/or shallower taper angle at the distal end of the balloon. The increased taper length and/or shallower taper angle at the distal end of the balloon may be as compared with the taper length and/or taper angle at the proximal end of the balloon.

In any of the described embodiments, the balloon may be less liable to prolapse when crossing a lesion due to increased taper length and/or shallower taper angle at the distal end of the balloon.

In any of the described embodiments, the first layer may be an outer layer, wherein an outer surface of the outer layer of the multilayer inflatable balloon includes a hydrophilic coating. The hydrophilic coating may be provided for improved lubricity.

In any of the described embodiments, compliance of the multilayer inflatable balloon at an inflation pressure of from about 2 atm to about 5 atm may be from about 0.5% to about 2% change in balloon diameter per atm, or no greater than about 1% change in balloon diameter per atm. In other words, when the balloon is inflated to a pressure of from about 2 atm to about 5 atm (e.g., 4 atm), any change in the balloon diameter may be minimal, with increased inflation pressure. For example, where the pressure is increased by 1 atm (e.g., to 5 atm), balloon diameter may only increase by about 0.5% to about 2%. Stated another way, where pressure is increased by 1 atm (e.g., to 5 atm), balloon diameter may increase by no more than about 1%.

In any of the described embodiments, the inner layer may be oriented at the maximum blow-up-ratio for the polymer material of that layer, and all successive layers may be oriented to less than the maximum blow-up-ratio for the polymer material comprising each such layer.

In any of the described embodiments, the multilayer inflatable balloon may have a hoop strength that is greater than that of a monolithic balloon of otherwise similar construction formed solely from a highest durometer material used in the multilayer inflatable balloon, having substantially the same wall thickness.

In any of the described embodiments, the combined wall thickness may be from about 0.01 mm to about 0.05 mm, or from about 0.01 mm to about 0.04 mm. Such thickness values may be for a thickness before inflation. Upon inflation, the thickness values may be about 5% to about 20% of such values. In another embodiment, e.g., for relatively low compliance balloon materials, the thickness values may be about 80-95% of such values, upon inflation.

In any of the described embodiments, the multilayer inflatable balloon may have an inflation diameter when inflated to about 2 atm to about 5 atm of from about 0.5 mm to about 15 mm. By way of example, a lithotripsy balloon catheter for coronary use may typically range from 0.8 mm to about 5 mm, or about 1 mm to about 5 mm in diameter. A lithotripsy balloon catheter for use with peripheral use may range from about 1.5 mm to about 14 mm in diameter.

In any of the described embodiments, the multilayer inflatable balloon may have an inflation length when inflated to about 2 atm to about 5 atm of from about 0.5 cm to about 30 cm. Exemplary balloon lengths may include 6 mm, 8 mm, 12 mm, 15 mm, 20 mm, 25 mm, 30 mm, 60 mm, or even as long as 250 mm or longer (e.g., for peripheral use).

In any of the described embodiments, the multilayer inflatable balloon may be pleated and compressed against the elongate catheter shaft so as to assume a reduced profile as a result of being compressed, as compared to a similar comparative balloon, that has not been compressed.

In any of the described embodiments, the multilayer inflatable balloon may exhibit improved damage resistance on an interior and/or exterior surface as compared to a comparative single layer balloon made of the first or second polymer material with a wall thickness equal or substantially equal to the combined wall thickness. One way to measure damage resistance may be in the ability of the balloon to be inflated and the electrode pulsed without rupture, for a given number of electrode pulse cycles (e.g., 160 or some other number of pulses). Other measures of damage resistance may relate to the resistance of the balloon to micro-dissections, spallation, scoring, tearing, thinning, or other damage. By way of example, the interior of the balloon may exhibit increased damage resistance, because two different materials can be employed, for the interior versus the exterior surface of the balloon. Increased interior damage resistance may be helpful as the interior surface is subject to plasma arcing, and other violent events during generation of the pressure waves. As to increased damage resistance of the exterior layer, this may be important in resisting damage from calcification. Calcium within the vasculature can be extensive and damaging to product used within that setting. Because the present lithotripsy catheters are designed to treat calcification, it would be a benefit and perceived advantage to have a balloon catheter that exhibits improved resistance to tearing or rupture related to contact with calcified vessel walls.

By way of example, a multilayer ballon structure including at least 4 layers may include a configuration that sandwiches the highly oriented layers (termed the inner and outer layers herein, which layers bear the pressure load) between a material that is not highly oriented (relative to its maximum blow up ratio, so as to provide a flexible protective layer on the inside and outside of the balloon. A significantly lower durometer hardness material (e.g., Durometer value of 20-30 Shore D) may be suitable for such. Another configuration could include at least 3 layers, where such a soft, flexible protective layer is only provided on the interior, or on the exterior of the multilayer balloon structure.

In any of the described embodiments, the multilayer inflatable balloon may exhibit improved dielectric properties as compared to a comparative single layer balloon made of the first or second polymer material with a wall thickness equal to the combined wall thickness.

In any of the described embodiments, the balloon catheter may be configured as a lithotripsy balloon catheter for use in heart valve lithotripsy or heart valve leaflet lithotripsy.

In any of the described embodiments, the inflation pressure during a given lithotripsy procedure may be no greater than about 4 atm.

In any of the described embodiments, between or after a given cycle of generating pressure waves for lithotripsy, the inflation pressure may be increased, e.g., to greater than about 10 atm, such as to between about 12 and about 16 atm, for example, to evaluate or measure the compliance of the vessel after such lithotripsy cycle.

In any of the described embodiments, where the lithotripsy emitter includes at least one electrode pair, the applied voltage may be from about 2000 V to 5000 V, or from about 2500 V to about 3500 V (e.g., about 3000 V). By way of example, such an electrode pair may be configured to receive high voltage pulses and generate a plasma arc within the inflation medium when the multilayer inflatable balloon is inflated.

In any of the described embodiments, the first layer may have a first Shore D durometer hardness and the second layer (e.g., the inner layer) may have a second Shore D durometer hardness lower than the first Shore D durometer hardness.

In any of the described embodiments, the multilayered balloon may have a higher rupture strength and/or a lower compliance as compared to a balloon of otherwise similar construction but formed solely from the highest Shore D durometer material used to form the multilayer inflatable balloon.

In any of the described embodiments, the inflation medium used to inflate the lithotripsy balloon may be a saline solution having an ionic strength sufficient to support generation of the plasma arc and resulting pressure wave. Where a laser is used as the lithotripsy emitter, the ionic strength of the solution may not be particularly important.

In any of the described embodiments, the generated pressure wave may exert a pressure of at least 30 atm, at least 35 atm, at least 40 atm, at least 45 atm, such as from 45 atm to 55 atm on the vessel wall, so as to break up the calcified tissue. In an embodiment, transmission of the pressure wave through the multilayer balloon may be substantially equal to transmission through a comparable single layer balloon of the same thickness, formed from either of the materials used in the multilayer balloon. For example, any loss attributable to the multilayer balloon may be less than about 25%, less than about 20%, less than about 15%, less than about 10%, less than about 5%, or less than about 3%, when comparing pressure values as transmitted through the multilayer balloon, as compared to a comparable single layer balloon.

In any of the described embodiments, the generated pressure wave may exert at least one of compressive, shearing, spallation, squeezing and/or cavitation forces on the vessel wall, so as to break up the calcified tissue.

In any of the described embodiments, where at least one electrode pair is employed as the lithotripsy emitter, the generated plasma arc may result in vaporization and/or cavitation within the inflation medium used to inflate the lithotripsy balloon, such vaporization and/or cavitation contributing at least in part to generation of the pressure wave. Where a laser lithotripsy emitter is employed, the laser energy itself may result in vaporization and/or cavitation within the inflation medium used to inflate the lithotripsy balloon, such vaporization and/or cavitation contributing at least in part to generation of the pressure wave. Use of a laser could conceivably also result in generation of a plasma arc, similar to an electrode pair. For example, the laser may be used to heat a metallic target, which upon heating generates a plasma, with an accompanying vaporization or cavitation bubble, or the like.

In any of the described embodiments, the vessel wall may include calcification extending from 270° to 360° about the vessel lumen.

In any of the described embodiments, the generated pressure wave may propagate through the inflation medium used to inflate the lithotripsy balloon, through the first and second layers of the multilayer inflatable balloon, and into the wall defining the vessel lumen, so as to break up the calcified tissue that is associated with the vessel wall.

In any of the described embodiments, the second maximum blow-up-ratio may be about 15 to about 40 percent greater than the first maximum blow-up-ratio.

In any of the described embodiments, the first maximum blow-up-ratio may be about 6 to about 7.

In any of the described embodiments, the second maximum blow-up-ratio may be about 7 to about 8.

In any of the described embodiments, the first maximum blow-up-ratio may be about 6 to about 7 and the second maximum blow-up-ratio may be about 7 to about 8.

In any of the described embodiments, the multilayer inflatable balloon may have a nominal working diameter corresponding to an inner mold diameter used to form the balloon, the first layer being substantially at the first maximum blow-up-ratio and the second layer being substantially at the second maximum blow-up-ratio when the multilayer inflatable balloon is substantially at the nominal working diameter.

In any of the described embodiments, the first layer may have a first Shore D durometer hardness and the second layer may have a second Shore D durometer hardness lower than the first Shore D durometer hardness.

In any of the described embodiments, the first polymer material may have a Shore D durometer hardness of about 70D to about 75D and the second polymer material may have a Shore D durometer hardness of about 60D to 65D.

In any of the described embodiments, the multilayered balloon may have a higher rupture strength and a lower compliance, compared to a balloon of otherwise similar construction (e.g., same or substantially same thickness) but formed solely from the highest Shore D durometer material used to form the multilayer inflatable balloon.

In any of the described embodiments, the multilayer inflatable balloon may have a nominal working diameter, wherein the balloon may have a noncompliant limited radial expansion beyond the nominal working diameter at pressures above a nominal pressure, up to a rated inflation burst pressure.

In any of the described embodiments, the balloon may have a higher modulus than a single layer balloon made of the first polymer material with a wall thickness equal to the combined wall thickness.

In any of the described embodiments, the first polymer material may be an elastomer.

In any of the described embodiments, the second polymer material may be an elastomer.

In any of the described embodiments, the first polymer material may include at least one of a polyamide, a polyurethane, a polyester, and/or a polyether block amide.

In any of the described embodiments, the second polymer material may include at least one of a polyamide, a polyurethane, a polyester, and/or a polyether block amide.

In any of the described embodiments, the first polymer material may include a polyether block amide having a first Shore D hardness and the second polymer material may include a polyether block amide having a different Shore D hardness.

In any of the described embodiments, the first layer may have a first elongation and the second layer may have a second elongation that is about 10 to about 50 percent more than the elongation of the first layer. Elongation of such materials may be measured by any suitable technique (e.g., ASTM D412 or otherwise).

In any of the described embodiments, the at least first and second layers collectively may define a rated inflation burst pressure from about 14 to about 22 atm when tested in a configuration where an outer surface of the balloon is not in apposition against any exterior supporting surface. It will be apparent that when in apposition against the exterior supporting vessel wall, the present multilayer balloons are able to withstand pressures associated with the described pressure wave (e.g., at least 30 atm, at least 35 atm, at least 40 atm at least 45 atm, such as from 45 to 55 atm during successive pressure wave events without exhibiting balloon failure).

In any of the described embodiments, the burst pressure of the at least first and second layers may be greater than a single layer balloon made of the first polymer material having a wall thickness equal to the combined wall thickness.

In any of the described embodiments, the multilayer inflatable balloon may be generally noncompliant with a compliance of less than about 0.03 mm/atm, or exhibit a compliance of about 0.5% to about 2% change in balloon diameter per atm, or no greater than about 1% change in balloon diameter per atm, when subjected to increased pressures between nominal pressure and a rated inflation burst pressure.

In any of the described embodiments, the first layer may be an outer layer, wherein the outer layer, inner layer, and any middle layer(s) may define a compliance less than that of a single layer balloon made of the polymer material of the outer layer polymer with a wall thickness equal to the combined wall thickness of the outer layer, inner layer, and middle layer(s).

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an exemplary general system for providing intravascular or other lithotripsy within a body lumen.

FIG. 2 illustrates an inflated balloon in a vessel for providing lithotripsy within such vessel.

FIG. 3 illustrates an exemplary multilayer, inflatable balloon catheter for providing lithotripsy procedures, embodying features of the present disclosure.

FIGS. 4 and 5 are transverse cross-sectional views of the catheter of FIG. 3.

FIG. 6 is a longitudinal cross-sectional view of multilayered balloon tubing in a balloon mold prior to being radially expanded therein, according to the present disclosure.

FIG. 7 illustrates an exemplary embodiment wherein an inner portion of a given balloon layer exhibits a higher degree of polymer chain orientation as compared to an outer portion of the given balloon layer.

FIG. 8 illustrates an exemplary multilayer, inflatable balloon catheter with an elongated tapered distal end for providing lithotripsy procedures, embodying features of the present disclosure.

FIG. 9 illustrates an exemplary multilayer, inflatable balloon catheter with an atraumatic, flexible tip for providing lithotripsy procedures, embodying features of the present disclosure.

FIGS. 10A-10B illustrate an exemplary multilayer, inflatable balloon catheter in a compressed and deflated configuration (FIG. 10B), and the same inflatable multilayer balloon catheter before being compressed using a balloon press (FIG. 10A).

FIG. 11 illustrates rupture strength test results for a dual layer balloon, versus a similarly sized single layer balloon.

### DETAILED DESCRIPTION

### I. Introduction

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

All publications and patent applications mentioned in this specification are herein incorporated by reference to the same extent as if each individual publication or patent application was specifically and individually indicated to be incorporated by reference, and as if each said individual publication or patent application was fully set forth, including any figures, herein. Two such patents that are incorporated herein by reference in their entirety are U.S. Pat. No. 7,828,766 and WO 2024/107418 (PCT/US2023/037234).

One or more embodiments of the present disclosure may generally relate to a balloon catheter including a multilayer balloon for use in a lithotripsy procedure.

### II. Definitions

For purposes of this disclosure, the following terms and definitions apply:

“Inflated diameter” or “expanded diameter” refers to the maximum diameter when the balloon is inflated to provide apposition to the interior vessel wall within a vessel. The inflated or expanded diameter typically does not result in any significant expansion of the vessel diameter during the lithotripsy portion of the procedure, which is in contrast to other types of balloon treatments (e.g., balloon angioplasty), which may inflate the balloon to a higher pressure, resulting in some expansion of the vessel diameter.

The terms “insulator”, “insulation” or “insulating” mean electrically insulating, and refer to dielectric materials that permit little, if any, flow of electrical current through such material. Insulating materials may also be thermally insulating but are not necessarily so. Materials such as glass, metal oxides, porcelain, paper, plastics, polymers, and rubbers are representative insulating materials.

The terms “conductor”, “conductive” and “conducting” mean electrically conductive, and refer to materials that easily permit the flow of electrical current through such material. Conductive materials may, in some examples, be thermally conductive, but are not necessarily so. Materials such as carbon black, graphite, gold, and other metals are representative conductive materials.

The phrase “conductive path” refers to a viable path for the flow of electrical current along such path and may, for example, refer to an entire surface of a balloon or component thereof coated with a conductive material, or only a portion of a surface coated with a conductive material such as conductive stripes or predetermined (e.g. rectangular) shapes on the surface or layer of a balloon. A conductive path may be placed on a surface of a balloon via known ink printing or other techniques. Examples of ink printed medical devices are described in U.S. Pat. Nos. 5,836,874; 7,379,767; 9,763,624; 9,913,594 and 10,751,000, each of which is herein incorporated by reference. In some instances, such a conductive path may create a virtual or actual faraday cage effect such that unwanted energy flow from the balloon to the patient is prevented.

The phrases “lithotripsy device” and “intravascular lithotripsy device” include devices that generate an acoustic pressure wave for use in a lithotripsy procedure. Such a pressure wave may be generated by using electrodes, providing arcing discharges between electrode components but may also include devices that create the acoustic pressure wave within the balloon via laser or optical energy, or use of other energy sources. Examples of such laser systems are described in U.S. Pat. Nos. 11,058,492 and 11,246,569, each of which is herein incorporated by reference in its entirety). Examples of electrode-based systems are described in U.S. Pat. Nos. 8,728,091; 9,642,673 and 10,850,078 and Published U.S. Pat. Appl. No. 2022-0054194, each of which is herein incorporated by reference in its entirety. Additional or alternative embodiments of electrode pairs are disclosed in U.S. Patent Application No. 63/735,842, filed Dec. 18, 2024 and entitled: INTRAVASCULAR LITHOTRIPSY CATHETER HAVING DIRECTIONAL ENERGY DELIVERY and PCT Publication No. WO2024/081361, the disclosure of each of which is herein incorporated by reference in its entirety.

The phrase “lithotripsy emitters” broadly includes electrode-based systems as well as laser or optical systems, or other energy sources for generating the desired acoustic pressure wave for use in lithotripsy.

The phrase “high voltage pulse” means an electrical pulse comprising a minimum voltage of at least 2000 volts, and in some embodiments encompasses a voltage of at least 3000 volts.

The phrase “thin-walled balloon material” means any compliant, semi-compliant or non-compliant balloon materials that are thin. Exemplary thicknesses may range from about 0.01 mm to about 0.04 mm. In an embodiment, the thin-walled balloon material comprises material that is in this thickness range in an unstretched state prior to inflation. One such material comprises a polyamide having repeating units linked by amide links. Some Nylon and PEBAX materials are suitable examples of such. The presently contemplated materials specifically include a multilayer structure. In some embodiments, thin-walled balloon materials may comprise balloon in balloon designs and the fluids filling the inner balloon may be dissimilar to the fluid filling the outer balloon to exploit differences in electrical insulative properties, speeds of sound of different materials, or the like. Exemplary compliant, semi-compliant, and non-compliant materials may include, but are not limited to nylon and other polyamides including polyether block amides, polyurethanes, silicones, polyesters (e.g., polyethylene terephthalate (PET)), and/or other biocompatible polymeric materials. In one embodiment, a burst pressure of exemplary thin-walled balloons may typically be at least 10 atm, such as from 14 atm to 22 atm. The thin-walled balloon is able to withstand interior pressures of very short duration that are greater than the burst pressure value associated with generation of the pressure wave, because the balloon is in apposition with the vessel wall during this active portion of the lithotripsy procedure.

The “blow-up-ratio (BUR)”, namely, the ratio of the outer diameter of the blown balloon (i.e., the mold inner diameter) to the inner diameter of the polymer tube from which the balloon is formed, is a measure of those dimensions. Beyond a critical BUR for a given polymer, the balloon blowing process becomes unstable and the polymer tubing often ruptures or tears before a balloon is fully formed.

The glass transition temperature (referred to herein as “Tg”) is the temperature at which the amorphous domains of a polymer change from a brittle vitreous state to a solid deformable or ductile state at atmospheric pressure. In other words, Tg corresponds to the temperature where the onset of segmental motion in the chains of the polymer occurs. Tg of a given polymer can be dependent on the heating rate and can be influenced by the thermal history of the polymer. Furthermore, the chemical structure of the polymer heavily influences the glass transition by affecting mobility of polymer chains.

“Stress” refers to force per unit area, as in the force acting through a small area within a plane within a subject material. Stress can be divided into components, normal and parallel to the plane, called normal stress and shear stress, respectively. Tensile stress, for example, is a normal component of stress that leads to expansion (increase in length) of the subject material. In addition, compressive stress is a normal component of stress resulting in compaction (decrease in length) of the subject material.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Modulus” may be defined as the ratio of a component of stress or force per unit area applied to a material divided by the strain along an axis of applied force that result from the applied force. For example, a material has both a tensile and a compressive modulus.

“Toughness” or “fracture toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough, is said to be brittle. Brittle materials are strong but cannot deform very much before breaking.

As used herein, the terms “axial” and “longitudinal” are used interchangeably and refer to a direction, orientation, or line that is parallel or substantially parallel to the central axis of the balloon or the central axis of a tubular construct (e.g., the balloon catheter). The term “circumferential” refers to the direction along a circumference of the balloon or tubular construct. The term “radial” refers to a direction, orientation, or line that is perpendicular or substantially perpendicular to the central axis of the balloon or the central axis of a tubular construct and may sometimes used to describe a circumferential property, i.e., radial strength.

### III. Exemplary Methods and Devices

The multilayered inflatable balloon catheter assemblies of the present disclosure may be configured for use in intravascular lithotripsy (IVL) procedures (e.g., within the coronary vasculature, or the peripheral vasculature), as well as other lithotripsy procedures, e.g., including but not limited to heart valve lithotripsy procedures and/or heart valve leaflet lithotripsy procedures. The present lithotripsy devices may be suitable for broad application within a wide variety of lithotripsy procedures.

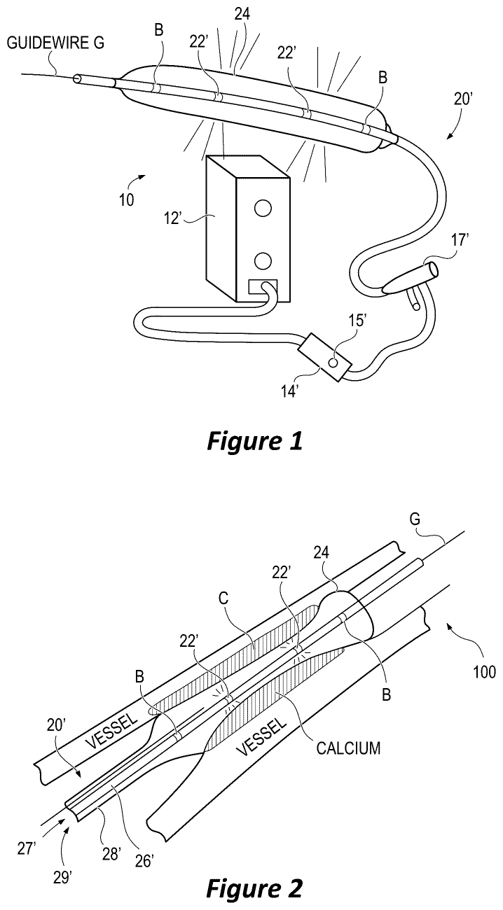

FIGS. 1-2 illustrates an exemplary lithotripsy device assembly. A lithotripsy device may comprise a power source 12′ (in the form of an electrical generator, but alternatively in the form of a laser system), a handle 14′ with therapy delivery control 15′ and a catheter 20′ with lithotripsy emitters 22′ (shown in the form of a pair of arcing electrodes, but alternatively they could comprise laser or other optical emitters configured to generate an acoustic pressure wave within the balloon), and a fluid filled balloon 24. Optional marker bands B may be provided. The catheter 20′ may include a central tube 26′ (FIG. 2) defining a guide wire lumen 27′ through which a guide wire G passes for delivering the balloon 24 at the desired location along the guide wire G. A sheath 28′ may surround the central tube 26′ and defines a delivery lumen 29′ through which saline or other inflation fluid can be controllably delivered for inflation of balloon 24. The lumen 29′ provides a concentric space around the central tube 26′ within which electrode or other power delivery wires (not shown) can be run from the control 15′ and/or power source 12′ to the lithotripsy emitters 22′ among other components in accordance with the present disclosure and discussed below. The sheath 28′ may be connected at a proximal end to a hub 17′ that can include any number of ports allowing electrode or other power delivery wires to pass into the lumen 29′ along with inflation fluid, the guide wire G, and any number of other components as desired.

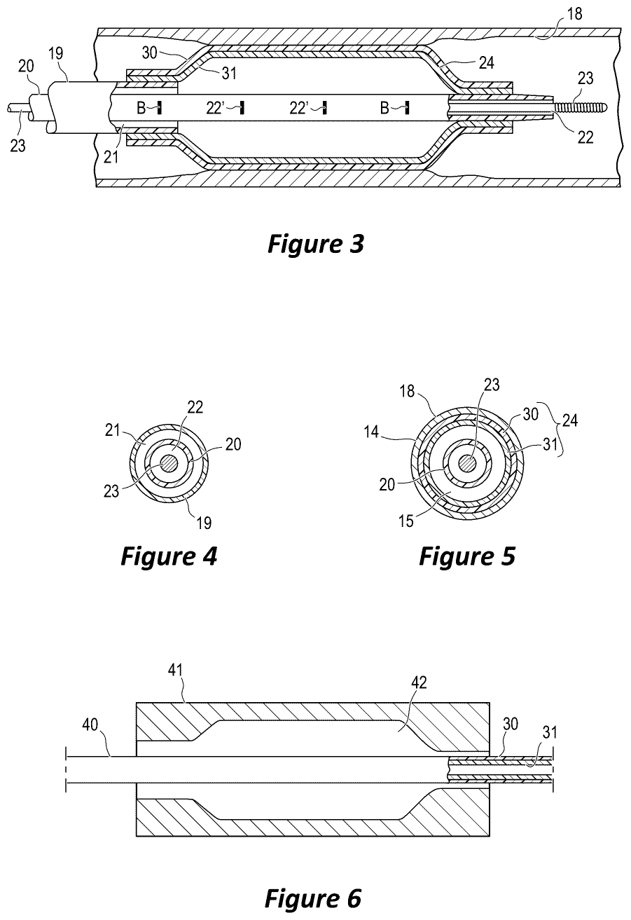

FIG. 2 shows the balloon 24 inflated to a therapy delivery state where the lithotripsy emitters 22′ may be “fired” to disrupt the calcium deposits within or on the vessel wall. Optional indicator bands B may be helpful in visualization and proper positioning relative to the calcified tissue to be treated, using known imaging techniques for such positioning. The balloon 24 is inflated to a typical lithotripsy pressure (e.g. 4 atm) and therapy is delivered. In some embodiments, the balloon 24 may naturally expand during or just after the therapy is delivered to open up the vessel. During actual delivery of the therapeutic acoustic pressure wave, the material of the balloon 24 is inflated to well below its burst pressure (e.g., 4 atm, as compared to 14-22 atm) to avoid balloon failure, and to ensure that the vessel is not significantly expanded, but that the balloon simply inflates to a sufficient degree to provide apposition against the interior vessel wall, but does not otherwise exert significant pressure on such vessel wall. Transmission of the acoustic pressure wave through the balloon and into the vessel wall will deliver at least one of compressive, shearing, spallation, squeezing and/or cavitation forces on the vessel wall, so as to disrupt the calcified tissue, but such forces are due to the acoustic pressure wave, not directly from any relatively high inflation pressure of the balloon itself. FIGS. 3-9 describe various embodiments of balloon materials including multi-layer balloons according to the present disclosure.

Balloons for conventional lithotripsy systems are constructed from a polymeric material, and are monolithic—i.e., comprised of a single layer of polymer. FIG. 3 illustrates an exemplary embodiment of a lithotripsy balloon catheter of the present disclosure, which differs in that the balloon layer is not monolithic, but is multilayered. Such a multilayered construction advantageously allows for improved rupture resistance to the balloon, and may provide one or more additional features, as well, as described herein.

FIG. 3 illustrates an exemplary multilayer balloon catheter in accordance with the present disclosure, with the multilayer balloon 24 in the inflated configuration with the balloon against the wall 18 of the body lumen. In the illustrated embodiment, the shaft comprises an outer tubular member 19 defining an inflation lumen 21, and an inner tubular member 20 defining the guidewire lumen 22 (e.g., configured to slidably receive a guidewire 23 therein) and positioned in the outer tubular member 19 such that the inflation lumen 21 is the annular space between the inner surface of the outer tubular member 19 and the outer surface of the inner tubular member 20, as perhaps best shown in FIG. 4, which illustrates a transverse cross-section of the catheter. The balloon 24 is illustrated as having a proximal skirt section sealingly secured to the distal end of the outer tubular member 19, and a distal skirt section sealingly secured to a distal end of the inner tubular member 20, so that an interior 15 of the balloon 24 is in fluid communication with the shaft 20′. The catheter shaft 20′ (shown in FIGS. 1 and 2) includes one or more lithotripsy emitters 22′ (e.g., a pair of arcing electrodes, but alternatively could comprise laser or other optical emitters), and optional marker bands B. The balloon catheter may comprise any desired number of emitters 22′. The catheter shaft 20′ is configured to provide sufficient power to the emitters 22′ within the multilayer inflatable balloon to generate an acoustic pressure wave within the inflation medium. By way of example, when using electrode emitters, the provided power may result in generation of a plasma arc, which generates the desired acoustic pressure wave. When using laser emitters, the provided power may result in vaporization and/or cavitation of the liquid inflation media (e.g., with use of an intermediate metallic or other target), which generates the desired acoustic pressure wave.

FIG. 5 illustrates a transverse cross-section of the catheter of FIG. 3, although the space between the inner surface of the balloon and the outer surface of the portion of the shaft 20 therein may be somewhat exaggerated in FIG. 5, for clarity. It will be apparent that a variety of alternative catheter shaft configurations can be used, employing a multilayer balloon as described herein.

Although not illustrated, the balloon 24 of the present disclosure may typically have a noninflated configuration with “wings” wrapped around the balloon to form a low profile configuration for introduction and advancement within a patient's body lumen. As a result, the balloon inflates to a nominal working diameter by unfolding and filling the molded volume of the balloon.

As illustrated in FIGS. 3 and 5, balloon 24 is comprised of a first layer 30 and a second layer 31 that is an inner layer relative to the first layer 30. The first and second layers 30, 31 have a combined wall thickness. The combined wall thickness may be from about 0.01 mm to about 0.04 mm. In the illustrated embodiment, the second layer 31 is on an inner surface of the first layer 30, with the first layer 30 defining an outer surface of the balloon 24 and the second layer 31 defining an inner surface of the balloon 24. However, the balloon 24 of the invention can alternatively have one or more additional layers, e.g., positioned on the interior of second layer 31, or on the exterior of first layer 30. Additional layer(s) increase the dimensions of the tube/balloon formed therefrom to a desired value, and/or can be used to provide an inner or outer surface of the balloon with a desired characteristic. For example, a relatively soft durometer material may be positioned interior to inner second layer 31, for improved durability (e.g., to better resist plasma arcing, or other violent characteristics present in such environment). A relatively soft durometer material may be positioned exterior to first layer 30 (e.g., to better resist damage or rupture of the balloon due to contact with calcified deposits within or on the vessel wall). It should be understood that the balloon 24 of the present disclosure has at least two layers, and optionally includes one or more additional layers. For example, the multilayer inflatable balloon 24 may have a total of from 2 to 5, from 2 to 4, or 2 or 3 layers (e.g., 2 layers, 3 layers, 4 layers or 5 layers).

In an embodiment, the first layer 30 (e.g., an outer layer) is formed of a first polymeric material, and the second layer 31 (an inner layer) is formed of a second polymeric material. The materials may be different, and may be specifically selected so that the second material can be expanded to a higher BUR than the first polymeric material. In an embodiment, the second layer 31 may be at a BUR that is about 15% to about 40% greater than the BUR of the first layer 30. In order to maximize strength, each of the first and second layers 30, 31 may be at its respective maximum BUR, so that the balloon includes multiple layers of highly oriented material and, consequently, a very low compliance. Alternatively, the inner layer may be oriented at the maximum blow-up-ratio for the polymer material of that layer, while all successive layers are oriented to less than the maximum blow-up-ratio for the polymer material comprising each such layer. The first maximum BUR (that of the first layer 30) may be from about 6 to about 7. The second maximum BUR (that of the second layer 31) may be about 7 to about 8.

The first and second layers 30, 31 may provide a rupture strength that is substantially equal to or greater than that of a comparative single layer (i.e., “monolithic”) balloon made solely of either the first or second polymeric material with a wall thickness equal to the combined wall thickness.

The densities of the polymers employed in the first and second layers 30, 31 of multilayer balloon 24 may be similar to one another. By way of example, the density of the second layer 31 may be within ±25%, ±20%, ±15%, ±10%, or ±5% of the density of the first layer 30. Such a substantial matching of densities may minimize any undesirable reflection of the acoustic pressure wave associated with the interface between such layers.

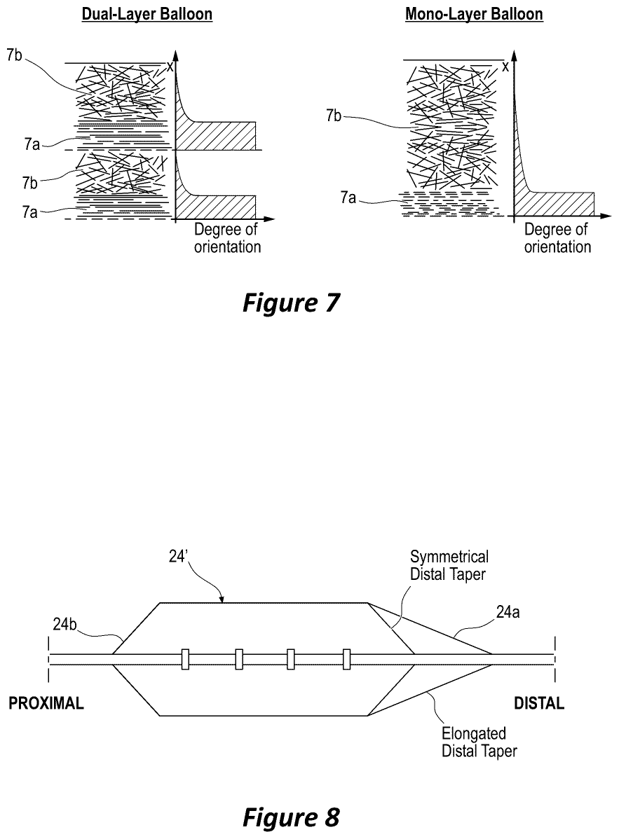

As shown in FIG. 7, each of the first and second layers 30, 31 may have a varying degree of polymer chain orientation as one moves from an inner surface of a given layer to an outer surface of such layer. More specifically, for each of the first and second layers 30, 31, the degree of polymer chain orientation may increase going from the outer surface of each layer to the inner surface of each layer. Such a gradient in the polymer chain orientation within each layer may result from the specific blow molding process used when forming the balloon. In such a case, an inner portion 7a of the first and/or second layers 30, 31 may exhibit a higher degree of polymer chain orientation as compared to an outer portion 7b of a given layer(s).

Such orientation of the polymer chains within the polymer layers may manifest itself as an increased degree of crystallinity within the inner portion of a given layer. By way of example, in an embodiment, the first and second layers 30, 31 may be configured such that the inner portion 7a of the inner layer (shown as second layer 31 in FIG. 3) and/or outer layer (shown as first layer 30 in FIG. 3) may exhibit a crystallinity that is within a range of about 30% to 100%, from about 35% to about 90%, or from about 40% to about 80%, while the outer portion 7b of the inner and/or outer layer may exhibit a lower crystallinity, e.g. that is within a range of 0% to about 30%, from about 0% to about 25%, or from about 0% to about 20%.

In an embodiment, the first layer may be thicker than the second layer. For example, the second layer 31 may account for about 10% to about 40% of the combined wall thickness of the multilayer inflatable balloon 24, while the first layer 30 may account for about 60% to about 90% of the combined wall thickness of the multilayer inflatable balloon 24.

The first and second layers 30, 31 may provide a rupture strength that is substantially equal to or greater than that of a comparative single layer balloon made solely of either the first or second polymer material with a wall thickness equal to the combined wall thickness of the multilayer balloon. What is particularly surprising is that the rupture strength of the balloon is greater than that of a monolithic balloon, made from the strongest (e.g., highest durometer) of the two materials.

The first and second layers 30, 31 may be selected from materials so as to exhibit different Shore D durometer hardness values. In an embodiment, the second layer 31 has a Shore D durometer hardness that is lower than the Shore D durometer hardness of the first layer 30. In other words, in such an embodiment, the Shore D durometer hardness of each layer of the multilayer balloon may increase from the inner most layer to the outer most layer of the multilayer balloon.

A variety of suitable materials can be used to form the first and second layers 30, 31, including, but not limited to polyamides, polyurethanes, polyesters, and/or polyether block amides. In an exemplary embodiment, the first and second polymeric materials are elastomers providing a relatively low flexural modulus for balloon flexibility. In other embodiments, some non-elastomers may alternatively be suitable for use. Particularly suitable materials are from the same polymeric family/class such as polyamides including Nylons and/or polyether block amides (e.g., PEBAX). Forming the layers from compatible polymeric materials allows for heat fusion bonding of the layers together. The layers can alternatively be formed of different polymer classes, even those that may not fusion bond together, e.g., where a tie layer may be provided between the outer and inner layers 30, 31 to bond the two balloon layers together. For example, a PET layer and a PEBAX layer may typically have a tie layer of an adhesive polymer such as Primacor (a functionalized polyolefin) therebetween.

In an embodiment, an outer layer of the balloon 24 (shown as first layer 30 in FIG. 3) may include a hydrophilic coating for increased lubricity of the balloon 24. Such a hydrophilic coating may be configured to render the surface of balloon 24 more slippery when in contact with bodily fluids, thus reducing friction and allowing for smoother insertion and manipulation during medical procedures. The hydrophilic coating may be incorporated into balloon 24 during extrusion of the balloon or by dip coating, spraying, or other suitable techniques, that will be apparent to those of skill in the art.

In an embodiment, the balloon 24 is formed by a method in which the layer(s) of material capable of expansion to higher BURs are the inner layer(s) of the balloon tubing, and lower BUR materials are the outer layer(s). The balloon is blow-molded such that each layer is optimized for radial expansion. Such a resulting balloon has an increased rupture resistance at increasing inflation pressures. Even at relatively low treatment pressures as employed in lithotripsy (e.g., no more than about 4-5 atm), the increased rupture resistance is beneficial, as the balloon may experience microdamage (e.g., micro-dissections, spallation, and/or other damage) to the balloon surface, during the lithotripsy procedure. The increased rupture resistance provided by use of a multilayer balloon structure advantageously reduces risk of rupture of the balloon, without any need to increase balloon wall thickness.

By way of example, the balloon 24 may be blow-molded from a multilayered polymeric tube including first and second layers 30, 31, where the second layer 31 is an inner layer relative to the first layer 30. As discussed above, a balloon of the present disclosure may optionally include one or more additional layer(s), so that the tubing used to blow-mold the balloon could similarly be formed with the additional layer(s). The tube may be formed by coextrusion, although a variety of alternative methods may be suitable for use, as well. In an embodiment, a multilayered tube is formed by coextruding at least two layers, and one or more additional layers may be added to the coextruded tube for example by heat shrinking, dip coating, adhesive or fusion bonding, or frictionally engaging the additional layer(s) to the coextruded tube. Alternatively, any number of desired layers may simply be coextruded together (e.g., 2, 3, 4, or 5 layers, coextruded together).

In any case, the multilayered tube may then be radially expanded in a balloon mold to form the balloon 24. FIG. 6 illustrates the multilayered tube 40 in a balloon mold 41 having an interior chamber 42 with a shape configured to form the balloon 24. Chamber 42 may have an inner diameter about equal to the nominal working diameter of the expanded balloon 24. The multilayered tube 40 may be stretched axially and heated during blow molding in the balloon mold, as appreciated by those of ordinary skill in the art. For example, in one embodiment, the tube is longitudinally stretched by about 200% during blow molding, which produces a biaxially oriented balloon. The wall thickness of the tube (prior to being radially expanded in the mold) may be about 0.1 mm to about 0.4 mm, and the wall thickness of the resulting uninflated balloon (after radially expanding in the mold) may be about 0.01 mm to about 0.04 mm, depending on the desired balloon characteristics and uses.

In an embodiment, the materials and dimensions of the multilayered tube 40, balloon mold 41, and chamber 42 are selected so that each layer of the resulting balloon has been radially expanded to substantially its maximum possible amount, expressed as the BUR of the balloon layers, during expansion in chamber 42. In an exemplary embodiment, the outer layer 30 has a higher Shore D durometer hardness and lower elongation than the one or more inner layer(s). In an embodiment, the first polymer material has a Shore D durometer hardness of about 70D to about 75D and the second polymer material has a Shore D durometer hardness of about 60D to 65D. In an embodiment, the elongation of each layer may be about 10% to about 50%, and more specifically about 20% more than the elongation of the outer layer immediately adjacent thereto. As such, in an embodiment, the second layer 31 may have an elongation that is about 10 to about 50 percent more than the elongation of the first layer 30. The multilayer inflatable balloon may have an inflation diameter of from about 0.5 mm to about 15 mm. By way of nonlimiting example, a lithotripsy balloon catheter for coronary use may typically have a balloon diameter ranging from 0.8 mm to about 5 mm, or about 1 mm to about 5 mm. A lithotripsy balloon catheter for peripheral use may have a diameter ranging from about 1.5 mm to about 14 mm. Inflation length for the balloon may be from about 0.5 cm to about 30 cm. Exemplary balloon lengths may include 6 mm, 8 mm, 12 mm, 15 mm, 20 mm, 25 mm, 30 mm, 60 mm, or even as long as 250 mm or longer (e.g., for peripheral use). Inflation pressures associated with such values may typically be from about 2 atm to about 5 atm (e.g., 4 atm).

In an exemplary embodiment, the first (outer) layer 30 comprises a PEBAX having a Shore D durometer hardness of about 72D, and the second (inner) layer 31 comprises a PEBAX having a Shore D durometer hardness of about 63D. By way of example, the PEBAX 72D outer layer 30 may have a BUR of between about 6 and about 7. Also by way of example, the 63D PEBAX inner layer 31 may have a BUR of between about 7 and about 8.

In an embodiment, a middle layer of intermediate BUR and/or durometer hardness may be provided between the outer and inner layers 30, 31. For example, in an embodiment, the balloon 24 has a first, outer layer 30 comprising 72D PEBAX, a second, inner layer 31 comprising 63D PEBAX, and a middle layer therebetween comprising 70D PEBAX. In an embodiment, the inner and middle layers may have a smaller wall thickness than the highest durometer layer of the balloon, and typically together may make up about 5% to about 25%, or 5% to 20%, or 10% to 15% of the total wall thickness of the multilayered balloon. The balloon 24 can similarly have one or more additional layers which similarly continue the pattern of sequentially decreasing BUR and/or increasing durometer from the inner toward the outer layers of the balloon. That said, in an embodiment, the balloon 24 may have a relatively soft outer-most layer having a Shore D durometer hardness less than the immediately adjacent inner layer of the balloon. This may provide for increased damage resistance to the exterior surface of the balloon when contacting calcified tissue, which may be sharp and/or abrasive. By way of example, such a relatively soft outer-most layer may have a relatively low Shore D durometer hardness of about 20D to about 55D, or 40D to about 55D. Exemplary Shore D durometer hardness values for such may include 20D, 25D, 30D, 35D, 40D, 45D, 50D, and 55D.

The multilayered balloon of the present disclosure may have a relatively high rupture strength (and associated rupture pressure), and low compliance, particularly when compared to a balloon of otherwise similar construction but formed solely of only one of the materials used to make the multilayered balloon of the invention (e.g., the highest durometer material used, such as a 72D PEBAX outer layer of multilayered balloon 24). Compliance is typically determined for the pressure range extending from the nominal pressure (i.e., the pressure required to fill the molded volume of the balloon to the blow-molded nominal diameter) to the burst pressure or the rated burst pressure of the balloon. The rated burst pressure (RBP), calculated from the average rupture pressure, is the pressure at which 99.9% of balloons can be pressurized to without rupturing, with 95% confidence.

By way of example, the multilayer balloon may have a rated inflation burst pressure that may be from about 14 atm to about 22 atm when tested in a configuration where an outer surface of the balloon is not in apposition against any exterior supporting surface. When in apposition against the inner surface of a vessel wall during a lithotripsy procedure, actual pressures associated with the acoustic pressure wave, which may be of very short duration, may be significantly higher (e.g., up to about 55 atm), and will not result in rupture of the balloon, e.g., due at least in part to apposition against the vessel wall. In any case, the burst pressure of the multilayer balloon may be greater than a comparable single layer balloon made solely from any of the polymer materials used in the multilayer balloon, having a wall thickness equal to the combined wall thickness of the multilayer balloon.

The multilayer inflatable balloon has a nominal working diameter corresponding to the inner mold diameter used to form the balloon. In an embodiment, each of both the first layer 30 and the second layer 31 may be substantially at their maximum BUR when the multilayer inflatable balloon is substantially at the nominal working diameter. The multilayered balloon 24 may have a nominal pressure (pressure applied during inflation in the mold) of from about 20 to about 35 atm. The applied pressure during lithotripsy use may be significantly lower, e.g., about 2 to about 5 atm, and more typically of about 3 to about 5 atm. In an embodiment, the balloon may have a RBP of about 14 to about 22 atm, more typically about 18 to about 20 atm. In an embodiment, the rupture pressure is typically about equal to, greater than, or not substantially less than (i.e., not more than about 5% to about 15% less than) a rupture pressure of a balloon of otherwise similar construction but formed solely from any of the polymeric materials used in forming the multilayer balloon (e.g., the highest durometer material).

In an embodiment, a multilayered balloon of the invention, for example having at least a 72D PEBAX outer layer and a 63D PEBAX inner layer, reaches the nominal diameter of the balloon at lithotripsy therapy inflation pressures of less than about 5 atm, and thereafter stretches in a noncompliant manner with a compliance of about 0.01 to about 0.02 mm/atm (e.g., less than about 1% increase in diameter per atm) within the rated pressure range (e.g., 8-20 atm) of the multilayered balloon. The multilayer inflatable balloon may have a noncompliant limited radial expansion (e.g., less than about 1% increase in diameter per atm) beyond the nominal working diameter at pressures above a nominal pressure.

Due to the presence of the softer durometer inner layer(s), the flexural modulus of a multilayered balloon of the invention is expected generally to be about 90% to about 95% of the flexural modulus of a balloon consisting of the first (e.g., higher durometer) elastomeric polymeric material of the layer 30. The multilayer balloon may have a higher modulus than a single layer balloon made solely of the first or second polymer material with a wall thickness equal to the combined wall thickness of the multilayer balloon.

In accordance with the present disclosure, the multilayer inflatable balloon may be generally noncompliant with a compliance of less than about 1% increase in diameter per atmosphere (e.g., less than about 0.03 mm/atm) between nominal pressure and a rated inflation burst pressure. In an embodiment, the outer layer, inner layer, and any middle layer(s) define a compliance less than that of a comparable single layer balloon made solely from the polymer material of the outer layer (or solely from any of the polymer material of any of the layers) with a wall thickness equal to the combined wall thickness of the outer layer, inner layer, and middle layer(s).

The multilayered inflatable balloon of the present disclosure may exhibit a hoop strength that is greater than that of a comparable, monolithic balloon of otherwise similar construction formed solely from the highest Shore D durometer material (or solely from any of the polymer materials) used in the multilayer inflatable balloon, having the same wall thickness as the combined wall thickness of the multilayer balloon.

In one embodiment, the multilayered balloon exhibits improved dielectric properties, as compared to a comparative single layer balloon made solely from the first or second polymer material with a wall thickness equal to the combined wall thickness of the multilayered balloon.

By way of example, an exemplary multilayered balloon tubing, formed by coextrusion, may have overall dimensions of 0.0180 inch (0.4572 mm) inner diameter (ID) and 0.0365 inch (0.9271 mm) outer diameter (OD). The tubing may have an inner layer of 63D PEBAX with a wall thickness of 0.0014 inch (0.03556 mm), and an outer layer of 72D PEBAX with a wall thickness of 0.00785 inch (0.19939 mm). Wall thickness values noted above are single wall thickness, unless otherwise identified as a double wall thickness (DWT). The tubing may be blow-molded by heating and pressurizing the tubing in a 0.1142 inch (2.90068 mm) ID balloon mold in a single blow cycle, resulting in a multilayered balloon having an average wall thickness (DWT) of 0.00165 inch (0.04191 mm) and the following BURs for the balloon layers: 63D Inner Layer ID of 0.0180 inch (0.4572 mm) gives a BUR of 6.34 (0.1142/0.0180); and 72 d outer layer ID of 0.0208 inch (0.52832 mm) gives a BUR of 5.49 (0.1142/0.0208). The resulting multilayered balloon has overall dimensions of about 0.1214 inch (3.08356 mm) ID and 0.1230 inch (3.1242 mm) OD. The OD of the balloon when blowing the balloon in the mold is equal to the ID of the mold, as the balloon is within the mold. The balloon OD post balloon blow is dependent on inflation pressure, as higher pressures result in increasing diameter values.

The rupture pressure (a measurement of rupture strength), compliance and modulus of the multilayered balloon may be compared to a comparison balloon similarly formed and with approximately the same wall thickness but from a single layer (100%) of the 72D PEBAX. Despite the presence of the lower durometer material in the inner layers, such that the 72D PEBAX makes up a smaller percentage of the wall thickness of the multilayer balloon as compared to the monolithic balloon made solely of 72D PEBAX, the multilayered balloon exhibits improved rupture strength. Specifically, the outer layer of 72D PEBAX may make up about 87% of the wall thickness of the multilayered balloon, compared to 100% of the monolithic balloon.

The BUR of the 72D PEBAX outer layer of the multilayer balloon is less than the BUR of the monolithic 72D PEBAX balloon. However, the multilayered balloon facilitates expanding the lower durometer inner layers to relatively high BURs, and provides a balloon with an overall BUR that is relatively high. The inner and middle layers are at relatively high BURs of about 7 to about 8, and preferably are at higher BURs than are possible if attempting to use the same blow-molding procedure to form a similar balloon but from 100% of the material of either the inner or the middle layer. For example, 63D PEBAX extruded to form tubing having an ID of 0.0195 inch (0.4953 mm) and an OD of 0.0355 inch (0.9017 mm) cannot be blown into a 0.118 inch (2.9972 mm) ID balloon mold (i.e., a BUR of 6) in a single blow cycle without rupturing during the blow-molding process. Alternative examples could be provided, using different materials (e.g., 72D PEBAX, 63D PEBAX, and/or Nylon 12 used in the various layers).

The absolute average combined wall thickness of the multilayered balloon in the above example is about equal to the wall thickness of the monolithic balloon. However, it should be understood that the combined wall thickness of the multilayered balloon of the invention could alternatively have been made less (while providing comparative rupture strength to the single layer monolithic balloon.

The dimensions of a balloon catheter in accordance with the present disclosure are determined largely by the size of the balloon and guidewire to be employed, the catheter type, and the size of the artery or other body lumen through which the catheter must pass. For example, the outer tubular member 19 may have an outer diameter of about 0.025 to about 0.04 inch (0.064 to 0.10 cm), for example, about 0.037 inch (0.094 cm), and the wall thickness of the outer tubular member 19 may vary from about 0.002 to about 0.008 inch (0.0051 to 0.02 cm), for example, about 0.002 to 0.005 inch (0.005 to 0.013 cm), or about 0.003 to 0.005 inch (0.0076 to 0.013 cm). The inner tubular member 20 may have an inner diameter of about 0.01 to about 0.018 inch (0.025 to 0.046 cm), for example, about 0.016 inch (0.04 cm), and a wall thickness of about 0.002 to about 0.004 inch (0.005 to 0.01 cm). The overall length of the catheter may range from about 100 to about 150 cm, for example, about 143 cm. Balloon 24 may have a length from about 0.5 cm to about 30 cm, such as 6 mm, 8 mm, 12 mm, 15 mm, 20 mm, 25 mm, 30 mm, 60 mm, or even as long as 250 mm or longer (e.g., for peripheral use), at an inflated lithotripsy pressure of about 2 to about 5 atm.

The various components may be joined together using conventional bonding methods such as by fusion bonding or use of adhesives. Although the shaft is illustrated as having an inner and outer tubular member, a variety of suitable shaft configurations may be used, including a dual lumen extruded shaft having a side-by-side lumens extruded therein. Similarly, although the embodiments described are an over-the wire type balloon catheter, the catheter of the present disclosure may comprise a variety of intravascular and other catheters, such as a rapid exchange type balloon catheters. Rapid exchange catheters generally comprise a shaft having a relatively short guidewire lumen extending from a guidewire distal port at the catheter distal end to a guidewire proximal port spaced a relatively short distance from the distal end of the catheter and a relatively large distance from the proximal end of the catheter.

During a lithotripsy procedure, the sonic pressure waves generate up to 55 atm of pressure during each pressure wave event. For example, the pressure wave may exhibit a pressure of at least 30 atm, or at least 35 atm, or at least 40 atm or at least 45 atm, such as from 45 to 55 atm. The multilayer balloon catheter of the present disclosure may be configured to withstand such pressures, when in apposition with the inner surface of the vessel wall, curing the lithotripsy procedure, without exhibiting balloon failure.

The inflation medium, the inner layer (shown as second layer 31 in FIG. 3), and/or the outer layer (shown as first layer 30 in FIG. 3) may be configured such that at least 50%, 55%, 60%, 65%, 70%, 75%, 80%, 85%, 90%, or 95% of the generated pressure wave is transmitted from the inflation medium into the inner layer and/or outer layer of the multilayer inflatable balloon 24. In other words, reflection of the acoustic pressure wave may be minimized, while transmission of the pressure wave through the various layers of the multilayer balloon and into the vessel wall may be maximized, so as to efficiently break up the calcified tissue. One might expect the addition of an interface between the two or more polymer layers of the multilayer balloon to potentially reduce transmission, and increase reflection of the acoustic pressure wave, although the embodiments of the present disclosures illustrate that high transmission can be achieved, providing a stronger balloon than a monolithic structure, that is still very effective in breaking up the target calcified deposits on or within the vessel wall.

The multilayer balloon catheter of the present disclosure may be configured such that any reflection associated with the interface between the inner layer (shown as second layer 31 in FIG. 3) and the outer layer (shown as first layer 30 in FIG. 3) (or an interface between any additional layers) is minimized, or of no significance. Measurement, calculation or other determination of transmission and/or reflection characteristics and values may be difficult, or may be accomplished in various ways. For example, it may not be practical to directly measure the transmission between individual balloon layers. However if transmission of the wave from the lithotripsy emitter or inflation medium (e.g., measuring the generated wave) to external of the balloon were measured such measurement could be used to inform the transmission through the balloon and associated material layers. For example, transmission through the multilayer balloon can be compared to transmission through a monolithic single layer balloon, showing that transmission is not significantly reduced, or is perhaps even improved. For example, when making such a comparison, measured pressure may not be attenuated in the multilayer balloon as compared to a single layer balloon by any more than about 25%, about 20%, about 15%, about 10%, about 5%, or about 3%.

As illustrated in FIG. 8, a balloon catheter in accordance with the present disclosure may include a taper at a distal end 24a of the balloon 24′ that differs from a taper at a proximal end 24b of the balloon 24′. The taper at the distal end 24a may provide a more gradual transition in radial width when crossing a lesion, as compared to the taper at the proximal end 24b of balloon 24′. In this way, the distal end 24a of the balloon may provide less crossing resistance when crossing a lesion due to an increased taper length and/or shallower taper angle at the distal end 24a of the balloon. By way of example only, the taper at the distal end 24a of the balloon may include a taper length of from about 3 mm to about 10 mm, and/or a taper angle of from about 10° to about 25°. In this embodiment, the balloon 24′ may be less liable to prolapse when crossing a lesion due to the increased taper length and/or shallower taper angle at the distal end 24a of the balloon 24′. While a more pronounced taper is shown at just the distal end, it will be appreciated that in an embodiment, a similarly pronounced taper may be provided at the proximal tip as well, should such be desired.

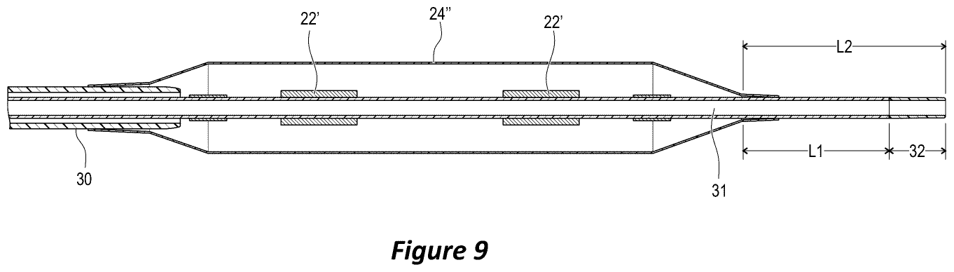

FIG. 9 illustrates an embodiment where a distal region of inner tubular member 20 extends, e.g., is extruded, beyond a distal end of the outer tubular member 19 to form an atraumatic, flexible tip 32. Towards this end, the balloon 24″ may comprise a cylindrical distal section having an overall length of L2, including the distal skirt section and distal tip 32. Without including the distal tip 32, the distal portion of the skirt section that surrounds, and is sealed, or bonded in a watertight engagement, against the outer surface of outer tubular member 19, comprises a length of L1, which is shorter than L2. Accordingly, the distal tip 32 extends distally beyond the distal end of the outer tubular member 19 for a distance to provide an atraumatic tip 32. Atraumatic tip 32 is preferably flexible and comprises a conduit defined therethrough that aligns with a conduit defined through the inner tubular member 20 to allow for, inter alia, guidewire access. Atraumatic, flexible tip 32 may facilitate translation through the vasculature. In this way, the multilayer inflatable balloon 24″ with atraumatic tip 32 may exhibit improved damage resistance on an interior surface of a vessel as compared to a similar balloon without an atraumatic tip. In an embodiment, such a flexible tip 32 may be formed using a “reflow” technique, where one or more of the polymer layers of the balloon are heated, and reflowed, to form such flexible tip 32. While the balloon includes multiple layers, in an embodiment, not all such layers may be reflowed (e.g., it may only be one or some of the polymer layers that are softened and reflowed, when forming such tip). Additional details of such a tip are disclosed in U.S. application Ser. No. 18/991,527 titled “INTRAVASCULAR LITHOTRIPSY SYSTEM WITH IMPROVED DURABILITY, EFFICIENCY AND PRESSURE OUTPUT VARIABILITY” filed Dec. 21, 2024, and U.S. application Ser. No. 18/991,526 titled “INTRAVASCULAR LITHOTRIPSY ALGORITHM FOR IMPROVED BALLOON DURABILITY” filed Dec. 21, 2024, each of which is herein incorporated by reference in its entirety.

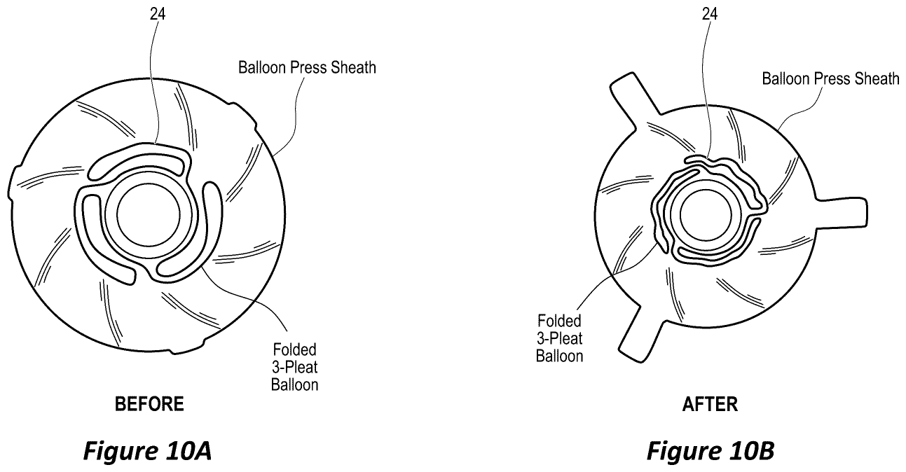

FIGS. 10A-10B illustrate how balloon 24 may be compressed during manufacture to reduce or minimize its radial dimension, prior to inflation. For example, as shown in FIG. 10A-10B, the multilayer inflatable balloon 24 may be pleated and compressed against the elongate catheter shaft so as to assume a reduced profile as a result of being compressed, as compared to a similar comparative balloon, that has not been compressed. In this way, the multilayer inflatable balloon 24 may exhibit improved damage resistance on an interior surface of a vessel as compared to a similar non-compressed balloon. For example, the pleated balloon may be folded to reduce radial profile (FIG. 10A), and then a balloon press (e.g., a sheath) may be fitted thereover, and used to compress the pleated and folded balloon, to reduce radial profile. Once compressed, a protective sheath may be placed over the compressed balloon, to maintain the reduced radial geometry (FIG. 10B). Such a method may include (1) providing a balloon catheter as described herein; (2) folding the multilayer layer balloon along one or more pleat lines, where the multilayer inflatable balloon is positioned about the elongate catheter shaft; (3) compressing the folded multilayer inflatable balloon using a balloon press; (4) removing the balloon press from about the multilayer inflatable balloon; and (5) positioning a balloon sheath over the compressed balloon to maintain a compressed geometry of the multilayer inflatable balloon.

The present disclosure also includes methods of using balloon catheters made in accordance with the above disclosure for IVL or other lithotripsy procedures. One embodiment of such a method includes: (1) providing a multilayer inflatable balloon catheter that is in accordance with any of the above disclosure; (2) inserting the multilayer inflatable balloon catheter into a vessel lumen to be treated, and inflating the multilayer inflatable balloon using the inflation medium, to a relatively low inflation pressure of no greater than about 5 atm (e.g., about 4 atm), so as to position the inflated multilayer inflatable balloon against an interior of a wall of the vessel lumen, in which the inflation pressure is sufficiently low so as to provide apposition against the vessel wall, but not to exert significant pressure against the vessel wall; and (3) applying power to the at least one lithotripsy emitter of the balloon catheter, so as to generate an acoustic pressure wave within the multilayer inflatable balloon, the pressure wave propagating through the inflation medium and through the thickness of the multilayer inflatable balloon and into the wall of the vessel lumen, so as to break up calcified tissue associated with the vessel wall.

According to the present disclosure, where the one or more lithotripsy emitters are configured as electrodes, the applied voltage during an exemplary lithotripsy procedure may be from about 2000 V to 5000 V, or from about 2500 V to about 3500 V (e.g., about 3000 V). Laser or other optical lithotripsy emitters may alternatively be used to generate the desired acoustic pressure wave. The inflation medium can be a saline solution, and may, e.g., be mixed with contrast media. An electrode generated plasma arc (or an intermediate target heated by the laser energy) may result in vaporization and/or cavitation within the inflation medium used to inflate the lithotripsy balloon and such vaporization and/or cavitation may contribute at least in part to generation of the pressure wave. The generated pressure wave may exert a pressure of at least 30 atm, at least 35 atm, at least 40 atm, at least 45 atm, such as from 45 atm to 55 atm on the vessel wall, so as to break up the calcified tissue. The generated pressure wave may exert at least one of compressive, shearing, spallation, squeezing and/or cavitation forces on the vessel wall, which aids in disrupting and breaking up the calcified tissue. The generated pressure wave propagates through the inflation medium used to inflate the multilayer inflatable balloon, through the first and second layers of the multilayer inflatable balloon, and into the wall defining the vessel lumen, so as to break up the calcified tissue that is associated with the vessel wall. The methods of the present disclosure may be particularly effective in treatment of calcified vasculature wherein the vessel wall includes calcification extending from 270° to 360° about the vessel lumen. That said, such procedures may also be effective, even where the calcification may extend to a lesser degree around the circumference of the vessel lumen.

While described principally in the context of intravascular lithotripsy procedures, it will be appreciated that the present devices and methods can be used within lithotripsy procedures more broadly. For example, such may also be used in heart valve and/or heart valve leaflet lithotripsy procedures. In an embodiment, such a method includes: (1) providing a multilayer inflatable lithotripsy balloon catheter in accordance with any of the above disclosure; (2) inserting the multilayer inflatable lithotripsy balloon catheter into a heart valve vessel lumen or heart valve leaflet vessel lumen to be treated, and inflating the multilayer inflatable lithotripsy balloon using the inflation medium, to a relatively low inflation pressure of no greater than about 5 atm, so as to position the inflated multilayer inflatable balloon against an interior of a wall of the vessel lumen, in which the inflation pressure is sufficiently low so as to provide apposition against the vessel wall, but not to exert significant pressure against the vessel wall; and (3) applying power to the at least one lithotripsy emitter, so as to generate a pressure wave within the multilayer inflatable balloon, the pressure wave propagating through the inflation medium and through the thickness of the multilayer inflatable balloon and into the wall of the vessel lumen, so as to break up calcified tissue associated with the vessel wall.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

Positioning of the “softer” layer on the inside of the balloon may be counterintuitive, given that one may expect such a soft inner layer to develop an imprint from the rigid electrodes within the balloon, during crimping. Such imprinting could potentially damage such inner layer of the balloon. With sufficient care in selection of materials, and care during crimping, such may not be problematic.

### IV. Comparative Test Data

Exemplary IVL balloon catheters including a dual layer balloon were tested for comparison against standard single layer balloon IVL catheters.

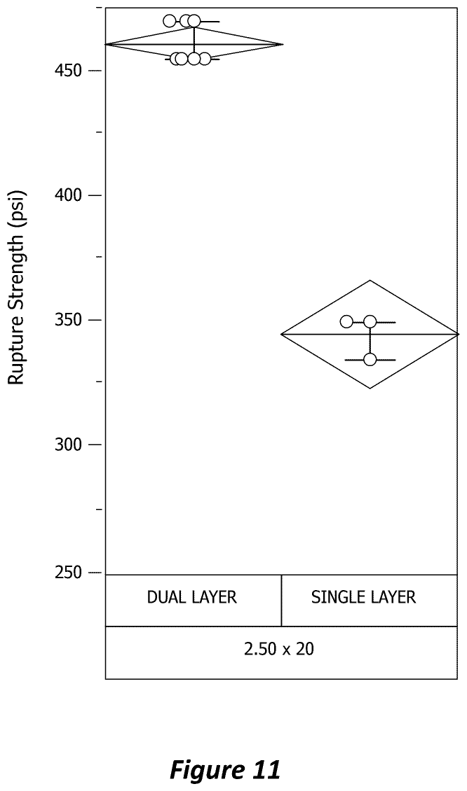

Prepared dual layer balloons were prepared measuring 2.5 mm in diameter with a length of 20 mm. The dual layer balloons were co-extruded, and formed so as to include a 72D PEBAX outer layer, and a 63D PEBAX inner layer. The dual layer balloons exhibit a rupture strength that is greater than a comparative single layer balloon formed solely from either 72D PEBAX or 63D PEBAX, with comparable total wall thickness, as shown in FIG. 11.

FIG. 11 shows rupture strength data for the dual layer balloons, as compared to an otherwise similar single layer balloon construction. As shown in FIG. 11, rupture strength increases significantly, for the dual layer balloon configuration, e.g., by about 100 psi or more, e.g., from about 350 psi for a single layer balloon to over 450 psi for the dual layer balloon, where both balloons measure 2.5 mm in diameter, and 20 mm in length and have substantially the same wall thickness. Such an increase in rupture strength is significant, representing a 20-30% or more increase in strength.

In addition to rupture testing, further testing was conducted to measure peak compressional pressure as delivered by an IVL electrode, through the dual layer balloon. For example, there may be some concern that due to the presence of such a dual layer structure and the associated interface between such layers, that there may be some unwanted reflection or loss of transmitted pressure through such a dual layer balloon, as compared to a single layer balloon. Four identical 2.5 mm×20 mm dual layer balloons, each including 2 electrode pairs, were tested by pulsing the electrodes repeatedly, to confirm that they would survive through 160 pulse cycles. The minimum, maximum, and average peak pressure values for each of the tested balloons, through the 160 pulse cycle test were measured. Results for such are shown in Table 1 below.

The results shown in Table 1 below indicate that the pressure wave is being efficiently transmitted through the dual layer balloon structure, with peak pressure values that are very similar to those measured with a single layer balloon. In other words, the presence of the dual layer balloon wall structure has little to no effect on pressure wave transmission, while at the same time providing increased strength and durability. For example, with a similarly sized and configured single layer balloon, the average peak pressure values are very similar (e.g., ranging from about 0.834 MPa to about 1.222 MPa), with similar standard deviation, minimum, and maximum peak pressure values as shown in Table 1. Such results indicate that use of a dual layer balloon provides advantageously increased strength, rupture resistance and durability as compared to a balloon of similar wall thickness. Alternatively, this allows for providing a balloon of similar strength and rupture resistance but with a thinner wall thickness (and improved crossing profile). The fact that all 4 tested dual layer balloons were able to withstand 160 pulse cycles is particularly beneficial, as often single layer balloons of similar thickness are not able to reach a threshold of 160 pulse cycles, without failure.

TABLE 1

| | Device 1 | Device 2 | Device 3 | Device 4 |
|---|---|---|---|---|
| Average (MPa) | 1.489 | 1.414 | 1.459 | 1.603 |
| Std. Dev. | 0.236 | 0.309 | 0.278 | 0.314 |
| Min. (MPa) | 0.940 | 0.892 | 0.891 | 1.016 |
| Max. (MPa) | 2.232 | 2.386 | 3.067 | 2.898 |

Test conditions for the data shown in Table 1 included preparation of each test device with a 50/50 saline/contrast inflation media. The test device was placed in a test fixture, in a vertical stand on a 0.014 inch (0.3556 mm) mandrel, with 2 hydrophones on opposing sides, positioned 5 mm from the central axis of the test device, with the hydrophones oriented towards the distal lithotripsy emitter of the test device, with the hydrophones aligned with each emitter window (i.e., centered on the spark event). The higher of the 2 data points recorded by the 2 hydrophones for each pulse were recorded. The test device was connected to filet, with the voltage generator programed to start at 2700 V, with a pre-programmed ramp of 25V increases every 10 pulses. The test device, fixture, and hydrophones were immersed in a 37° C. water bath, and each test device was pulsed to 160 pulse cycles, in 10 pulse increments, with degassing after every 10 pulses (e.g., degassing via a vacuum syringe attached to 3-way stopcock, with vacuum applied during 10-30 second degas step).

Any ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, or “substantially” as used herein include the recited numbers (e.g., about 10% includes 10%), and also represent an amount close to the stated amount that still performs a desired function or achieves a desired result. For example, the terms “approximately”, “about”, and “substantially” may refer to an amount that is within 10% of, within 5% of, within 1% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Reference to ASTM or other standardized tests refers to the latest version of such standard, unless otherwise specified. Standards referenced herein are herein incorporated by reference in their entirety.

Following are some further example embodiments of the invention. These are presented only by way of example and are not intended to limit the scope of the invention in any way. Further, any example embodiment can be combined with one or more of the example embodiments.

Embodiment 1. A balloon catheter for use in a lithotripsy procedure, comprising: an elongate catheter shaft; an inflation lumen; a multilayer inflatable balloon in fluid communication with the inflation lumen located toward, or at, a distal section of the elongate catheter shaft; and at least one lithotripsy emitter within the multilayer inflatable balloon capable of generating an acoustic pressure wave within an inflation medium when the multilayer inflatable balloon is inflated; wherein the multilayer inflatable balloon comprises: a first layer and a second layer having a combined wall thickness; the first layer being made of a first polymer material; the second layer being made of a second polymer material; wherein the second layer is an inner layer relative to the first layer. In an embodiment, the multilayer balloon may exhibit a rupture strength that is greater than a comparative single layer balloon formed solely from either the first polymer material or the second polymer material with a wall thickness equal to the combined wall thickness.

Embodiment 2. The balloon catheter of embodiment 1, wherein multilayer balloon exhibits a rupture strength that is at least 5% greater than a comparative single layer balloon formed solely from either the first polymer material or the second polymer material with a wall thickness equal to the combined wall thickness.

Embodiment 3. The balloon catheter of embodiment 1 or 2, wherein the at least one lithotripsy emitter comprises a pair of electrodes, wherein the catheter is configured to provide sufficient power to the electrodes within the multilayer inflatable balloon to generate a plasma arc within the inflation medium used to inflate the multilayer inflatable balloon, and generation of the plasma arc resulting in a pressure wave within the multilayer inflatable balloon during the lithotripsy procedure.

Embodiment 4. The balloon catheter of any of the preceding embodiments, wherein the first and second layers provide a rupture strength that is substantially equal to or greater than that of a comparative single layer balloon made of the first polymer material with a wall thickness equal to the combined wall thickness.

Embodiment 5. The balloon catheter of any of the preceding embodiments, wherein the first and second layers provide a rupture strength that is substantially equal to or greater than that of a comparative single layer balloon made of the second polymer material with a wall thickness equal to the combined wall thickness.

Embodiment 6. The balloon catheter of any of the preceding embodiments, wherein a density of the second layer is within ±25%, ±20%, ±15%, ±10%, or ±5% of a density of the inner layer.

Embodiment 7. The balloon catheter of any of the preceding embodiments, wherein an inner portion of the inner layer exhibits a higher degree of polymer chain orientation as compared to an outer portion of the inner layer.

Embodiment 8. The balloon catheter of any of the preceding embodiments, wherein the first layer is an outer layer, wherein an inner portion of the outer layer exhibits a higher degree of polymer chain orientation as compared to an outer portion of the outer layer.

Embodiment 9. The balloon catheter of any of the preceding embodiments, wherein the first layer is an outer layer, wherein an inner portion of the inner layer exhibits a higher degree of polymer chain orientation as compared to an outer portion of the inner layer, and an inner portion of the outer layer exhibits a higher degree of polymer chain orientation as compared to an outer portion of the outer layer.

Embodiment 10. The balloon catheter of any of the preceding embodiments, wherein the inner layer accounts for about 10% to about 40% of the combined wall thickness of the multilayer inflatable balloon.

Embodiment 11. The balloon catheter of any of the preceding embodiments, wherein the first layer is an outer layer, wherein the outer layer accounts for about 60% to about 90% of the combined wall thickness of the multilayer inflatable balloon.

Embodiment 12. The balloon catheter of any of the preceding embodiments, wherein the multilayer inflatable balloon includes a total of from 2 to 5, from 2 to 4, or 2 or 3 layers.

Embodiment 13. The balloon catheter of any of the preceding embodiments, wherein the multilayer inflatable balloon includes only two layers.

Embodiment 14. The balloon catheter of any of the preceding embodiments, wherein the at least one lithotripsy emitter includes at least two electrode pairs (i.e., 4 electrodes).

Embodiment 15. The balloon catheter of any of the preceding embodiments, wherein the at least one lithotripsy emitter includes a laser or other optical emitter.

Embodiment 16. The balloon catheter of any of the preceding embodiments, wherein the multilayer inflatable balloon includes a taper at a distal end of the balloon that differs from a taper at a proximal end of the balloon.

Embodiment 17. The balloon catheter of embodiment 16, wherein the taper at the distal end of the balloon provides a more gradual transition in radial width when crossing a lesion, as compared to the taper at the proximal end of the balloon.

Embodiment 18. The balloon catheter of embodiment 16 or 17, wherein the taper at the distal end of the balloon includes a taper length of from about 3 mm to about 10 mm, and/or a taper angle of from about 10° to about 25°.

Embodiment 19. The balloon catheter of embodiment 18, wherein the distal end of the balloon provides less crossing resistance when crossing a lesion due to increased taper length and/or shallower taper angle at the distal end of the balloon.

Embodiment 20. The balloon catheter of embodiment 18 or 19, wherein the balloon is less liable to prolapse when crossing a lesion due to increased taper length and/or shallower taper angle at the distal end of the balloon.

Embodiment 21. The balloon catheter of any of the preceding embodiments, wherein the first layer is an outer layer, wherein an outer surface of the outer layer of the multilayer inflatable balloon includes a hydrophilic coating for improved lubricity.

Embodiment 22. The balloon catheter of any of the preceding embodiments, wherein compliance of the multilayer inflatable balloon at an inflation pressure of from about 2 atm to about 5 atm is less than about 1% per atm.

Embodiment 23. The balloon catheter of any of the preceding embodiments, wherein the inner layer is oriented at a maximum blow-up-ratio for the polymer material of that layer, and all successive layers are oriented to less than a maximum blow-up-ratio for the polymer material comprising each such layer.

Embodiment 24. The balloon catheter of any of the preceding embodiments, wherein the multilayer inflatable balloon has a hoop strength that is greater than that of a monolithic balloon of otherwise similar construction formed solely from a highest durometer material used in the multilayer inflatable balloon, having the same wall thickness.

Embodiment 25. The balloon catheter of any of the preceding embodiments, wherein the combined wall thickness is from about 0.01 mm to about 0.04 mm.

Embodiment 26. The balloon catheter of any of the preceding embodiments, wherein the multilayer inflatable balloon has an inflation diameter when inflated to about 2 atm to about 5 atm that is from about 0.5 mm to about 15 mm.

Embodiment 27. The balloon catheter of any of the preceding embodiments, wherein the multilayer inflatable balloon has an inflation length when inflated to about 2 atm to about 5 atm that is from about 0.5 cm to about 30 cm.

Embodiment 28. The balloon catheter of any of the preceding embodiments, wherein the multilayer inflatable balloon is pleated and compressed against the elongate catheter shaft so as to assume a reduced profile as a result of being compressed, as compared to a similar comparative balloon, that has not been compressed.

Embodiment 29. The balloon catheter of any of the preceding embodiments, wherein the multilayer inflatable balloon exhibits improved damage resistance on an interior surface as compared to a comparative single layer balloon made solely from the first or second polymer material with a wall thickness equal to the combined wall thickness.

Embodiment 30. A balloon catheter for use in a lithotripsy procedure, comprising: an elongate catheter shaft; an inflation lumen; a multilayer inflatable balloon in fluid communication with the inflation lumen located toward, or at, a distal section of the elongate catheter shaft; an at least one lithotripsy emitter within the multilayer inflatable balloon capable of generating an acoustic pressure wave within an inflation medium when the multilayer inflatable balloon is inflated; wherein the multilayer inflatable balloon comprises: a first layer and a second layer having a combined wall thickness; the first layer being made of a first polymer material having a first maximum blow-up-ratio; the second layer being made of a second polymer material having a second maximum blow-up-ratio greater than the first maximum blow-up-ratio; and wherein the second layer is an inner layer relative to the first layer.

Embodiment 31. The balloon catheter of embodiment 30, wherein the balloon catheter is configured as a lithotripsy balloon catheter for use in intravascular lithotripsy.

Embodiment 32. The balloon catheter of embodiment 30 or 31, wherein the balloon catheter is configured as a lithotripsy balloon catheter for use in heart valve lithotripsy or heart valve leaflet lithotripsy.

Embodiment 33. A method of manufacturing a balloon catheter, the method comprising: providing a balloon catheter comprising: an elongate catheter shaft and an inflation lumen; a multilayer inflatable balloon in fluid communication with the inflation lumen located toward, or at, a distal section of the elongate catheter shaft; and at least one lithotripsy emitter within the multilayer inflatable balloon capable of generating an acoustic pressure wave within an inflation medium when the multilayer inflatable balloon is inflated; wherein the multilayer inflatable balloon comprises: a first layer and a second layer having a combined wall thickness; the first layer being made of a first polymer material; the second layer being made of a second polymer material, wherein the second layer is an inner layer relative to the first layer; folding the multilayer layer balloon along one or more pleat lines, where the multilayer inflatable balloon is positioned about the elongate catheter shaft; compressing the folded multilayer inflatable balloon using a balloon press; removing the balloon press from about the multilayer inflatable balloon; and positioning a balloon sheath over the compressed balloon to maintain a compressed geometry of the multilayer inflatable balloon.

Embodiment 34. The method of embodiment 33, wherein the first and second layers provide a rupture strength that is substantially equal to or greater than that of a comparative single layer balloon made of the first polymer material with a wall thickness equal to the combined wall thickness.

Embodiment 35. The method of embodiment 33 or 34, wherein the first and second layers provide a rupture strength that is substantially equal to or greater than that of a comparative single layer balloon made of the second polymer material with a wall thickness equal to the combined wall thickness.

Embodiment 36. A method for using a balloon catheter in a lithotripsy procedure, the method comprising: providing a lithotripsy balloon catheter comprising: an elongate catheter shaft having a proximal section and a distal section; an inflation lumen; a multilayer inflatable balloon located toward, or at, a distal section of the elongate catheter shaft; and at least one lithotripsy emitter within the multilayer inflatable balloon capable of generating an acoustic pressure wave within an inflation medium when the multilayer inflatable balloon is inflated during use; wherein the multilayer inflatable balloon comprises: at least a first layer and a second layer having a combined wall thickness; the first layer being made of a first polymer material; the second layer being made of a second polymer material, wherein the second layer is an inner layer relative to the first layer; inserting the lithotripsy balloon catheter into a vessel lumen to be treated, and inflating the multilayer balloon using the inflation medium, to a relatively low inflation pressure of no greater than about 5 atm, so as to position the inflated multilayer inflatable balloon against an interior of a wall of the vessel lumen, in which the inflation pressure is sufficiently low so as to provide apposition against the vessel wall, but not to exert significant pressure against the vessel wall; and applying power to the at least one lithotripsy emitter, so as to generate an acoustic pressure wave within the multilayer inflatable balloon, the pressure wave propagating through the inflation medium and through the thickness of the multilayer inflatable balloon and into the wall of the vessel lumen, so as to break up calcified tissue associated with the vessel wall.

Embodiment 37. The method of embodiment 36, wherein the first and second layers provide a rupture strength that is substantially equal to or greater than that of a comparative single layer balloon made of the first polymer material with a wall thickness equal to the combined wall thickness.

Embodiment 38. The method of any of embodiments 36 or 37, wherein the first and second layers provide a rupture strength that is substantially equal to or greater than that of a comparative single layer balloon made of the second polymer material with a wall thickness equal to the combined wall thickness.

Embodiment 39. The method of any of embodiments 36 to 38, wherein the inflation pressure is no greater than about 4 atm.

Embodiment 40. The method of any of embodiments 36 to 39, wherein the at least one lithotripsy emitter comprises at least one electrode pair, wherein an applied voltage is from about 2000 V to 5000 V, or from about 2500 V to about 3500 V (e.g., about 3000 V).

Embodiment 41. The method of any of embodiments 36 to 40, wherein the first layer has a first Shore D durometer hardness and the second layer has a second Shore D durometer hardness lower than the first Shore D durometer hardness.

Embodiment 42. The method of embodiment 41, wherein the multilayered balloon has a higher rupture strength and a lower compliance as compared to a balloon of otherwise similar construction and substantially equal thickness but formed solely from the highest Shore D durometer material used to form the multilayer inflatable balloon.

Embodiment 43. The method of any of embodiments 36 to 42, wherein the generated pressure wave exerts a pressure of at least 30 atm, at least 35 atm, at least 40 atm, at least 45 atm, such as from 45 atm to 55 atm on the vessel wall, so as to break up the calcified tissue.

Embodiment 44. The method of any of embodiments 36 to 43, wherein the generated pressure wave exerts at least one of compressive, shearing, spallation, squeezing or cavitation forces on the vessel wall, so as to break up the calcified tissue.

Embodiment 45. The method of any of embodiments 36 to 44, wherein the at least one lithotripsy emitter generates a plasma arc that results in vaporization and/or cavitation within the inflation medium used to inflate the lithotripsy balloon, such vaporization and/or cavitation contributing at least in part to generation of the pressure wave.

Embodiment 46. The method of any of embodiments 36 to 45, wherein the vessel wall includes calcification extending from 270° to 360° about the vessel lumen.

Embodiment 47. The method of any of embodiments 36 to 46, wherein the generated pressure wave propagates through the inflation medium used to inflate the lithotripsy balloon, through the first and second layers of the multilayer inflatable balloon, and into the wall defining the vessel lumen, so as to break up the calcified tissue that is associated with the vessel wall.

Embodiment 48. A method for using a balloon catheter in a heart valve or heart valve leaflet lithotripsy procedure, the method comprising: providing a lithotripsy balloon catheter comprising: an elongate catheter shaft having a proximal section and a distal section; an inflation lumen; a multilayer inflatable balloon located toward, or at, a distal section of the elongate catheter shaft; and at least one lithotripsy emitter within the multilayer inflatable balloon capable of generating an acoustic pressure wave within an inflation medium when the multilayer inflatable balloon is inflated during use; wherein the multilayer inflatable balloon comprises: at least a first layer and a second layer having a combined wall thickness; the first layer being made of a first polymer material; the second layer being made of a second polymer, wherein the second layer is an inner layer relative to the first layer; inserting the lithotripsy balloon catheter into a heart valve vessel lumen or heart valve leaflet vessel lumen to be treated, and inflating the lithotripsy balloon using the inflation medium, to a relatively low inflation pressure of no greater than about 5 atm, so as to position the inflated multilayer inflatable balloon against an interior of a wall of the vessel lumen, in which the inflation pressure is sufficiently low so as to provide apposition against the vessel wall, but not to exert significant pressure against the vessel wall; and applying power to the at least one lithotripsy emitter, so as to generate an acoustic pressure wave within the multilayer inflatable balloon, the pressure wave propagating through the inflation medium and through the thickness of the multilayer inflatable balloon and into the wall of the vessel lumen, so as to break up calcified tissue associated with the vessel wall.

Embodiment 49. The method of embodiment 48, wherein the first and second layers provide a rupture strength that is substantially equal to or greater than that of a comparative single layer balloon made of the first polymer material with a wall thickness equal to the combined wall thickness.

Embodiment 50. The method of embodiment 48 or 49, wherein the first and second layers provide a rupture strength that is substantially equal to or greater than that of a comparative single layer balloon made of the second polymer material with a wall thickness equal to the combined wall thickness.

Embodiment 51. The balloon catheter of any of the preceding embodiments, wherein a second maximum blow-up-ratio of the second polymer is about 15 to about 40 percent greater than a first maximum blow-up-ratio of the first polymer.

Embodiment 52. The balloon catheter of any of the preceding embodiments, wherein a first maximum blow-up-ratio of the first polymer is about 6 to about 7.

Embodiment 53. The balloon catheter of any of the preceding embodiments, wherein a second maximum blow-up-ratio of the second polymer is about 7 to about 8.

Embodiment 54. The balloon catheter of any of the preceding embodiments, wherein a first maximum blow-up-ratio of the first polymer is about 6 to about 7 and a second maximum blow-up-ratio of the second polymer is about 7 to about 8.

Embodiment 55. The balloon catheter of any of the preceding embodiments, wherein the first layer has a first Shore D durometer hardness and the second layer has a second Shore D durometer hardness lower than the first Shore D durometer hardness.

Embodiment 56. The balloon catheter of embodiment 55, wherein the first polymer material has a Shore D durometer hardness of about 70D to about 75D and the second polymer material has a Shore D durometer hardness of about 60D to 65D.

Embodiment 57. The balloon of embodiment, wherein the multilayered balloon has a higher rupture strength and a lower compliance, compared to a balloon of otherwise similar construction and thickness but formed solely from the highest Shore D durometer material used to form the multilayer inflatable balloon.

Embodiment 58. The balloon catheter of any of the preceding embodiments, wherein the balloon has a higher modulus than a single layer balloon made solely from the first or second polymer material with a wall thickness equal to the combined wall thickness.

Embodiment 59. The balloon catheter of any of the preceding embodiments, wherein the first polymer material is an elastomer.

Embodiment 60. The balloon catheter of any of the preceding embodiments, wherein the second polymer material is an elastomer.

Embodiment 61. The balloon catheter of any of the preceding embodiments, wherein the first polymer material comprises at least one of a polyamide, a polyurethane, a polyester, or a polyether block amide.

Embodiment 62. The balloon catheter of any of the preceding embodiments, wherein the second polymer material comprises at least one of a polyamide, a polyurethane, a polyester, or a polyether block amide.

Embodiment 63. The balloon catheter of any of the preceding embodiments, wherein the first polymer material comprises a polyether block amide having a first Shore D hardness and the second polymer material comprises a polyether block amide having a different Shore D hardness.

Embodiment 64. The balloon catheter of any of the preceding embodiments, wherein the first layer has a first elongation and the second layer has a second elongation that is about 10 to about 50 percent more than the elongation of the first layer.

Embodiment 65. The balloon catheter of any of the preceding embodiments, wherein the at least first and second layers collectively define a rated inflation burst pressure from about 14 to about 22 atm when tested in a configuration where an outer surface of the balloon is not in apposition against any exterior supporting surface.

Embodiment 66. The balloon catheter of embodiment 65, wherein the burst pressure of the at least first and second layers is greater than a single layer balloon made of the first polymer material having a wall thickness equal to the combined wall thickness.

Embodiment 67. The balloon catheter of any of the preceding embodiments, wherein the multilayer inflatable balloon is generally noncompliant with a compliance of less than about 1% per atmosphere between nominal pressure and a rated inflation burst pressure.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A balloon catheter for use in a lithotripsy procedure, comprising:
an elongate catheter shaft;
an inflation lumen;
a multilayer inflatable balloon in fluid communication with the inflation lumen located toward, or at, a distal section of the elongate catheter shaft; and
at least one lithotripsy emitter within the multilayer inflatable balloon capable of generating an acoustic pressure wave within an inflation medium when the multilayer inflatable balloon is inflated;
wherein the multilayer inflatable balloon comprises:
a first layer and a second layer having a combined wall thickness;
the first layer being made of a first polymer material;
the second layer being made of a second polymer material;
wherein the second layer is an inner layer relative to the first layer;
wherein multilayer balloon exhibits a rupture strength that is greater than a comparative single layer balloon formed solely from either the first polymer material or the second polymer material with a wall thickness equal to the combined wall thickness.

2. (canceled)

3. The balloon catheter of claim 1, wherein the at least one lithotripsy emitter comprises a pair of electrodes, wherein the catheter is configured to provide sufficient power to the electrodes within the multilayer inflatable balloon to generate a plasma arc within the inflation medium used to inflate the multilayer inflatable balloon, and generation of the plasma arc resulting in a pressure wave within the multilayer inflatable balloon during the lithotripsy procedure.

4. (canceled)

5. (canceled)

6. The balloon catheter of claim 1, wherein a density of the second layer is within ±25%, ±20%, ±15%, ±10%, or ±5% of a density of the inner layer.

7. (canceled)

8. (canceled)

9. The balloon catheter of claim 1, wherein the first layer is an outer layer, wherein an inner portion of the inner layer exhibits a higher degree of polymer chain orientation as compared to an outer portion of the inner layer, and an inner portion of the outer layer exhibits a higher degree of polymer chain orientation as compared to an outer portion of the outer layer.

10. The balloon catheter of claim 1, wherein the inner layer accounts for about 10% to about 40% of the combined wall thickness of the multilayer inflatable balloon.

11. (canceled)

12. The balloon catheter of claim 1, wherein the multilayer inflatable balloon includes a total of from 2 to 5, from 2 to 4, or 2 or 3 layers.

13. (canceled)

14. The balloon catheter of claim 1, wherein the at least one lithotripsy emitter includes at least two electrode pairs (i.e., 4 electrodes).

15. The balloon catheter of claim 1, wherein the at least one lithotripsy emitter includes a laser or other optical emitter.

16. The balloon catheter of claim 1, wherein the multilayer inflatable balloon includes a taper at a distal end of the balloon that differs from a taper at a proximal end of the balloon.

17. The balloon catheter of claim, wherein the taper at the distal end of the balloon provides a more gradual transition in radial width when crossing a lesion, as compared to the taper at the proximal end of the balloon.

18. The balloon catheter of claim, wherein the taper at the distal end of the balloon includes a taper length of from about 3 mm to about 10 mm, and/or a taper angle of from about 10° to about 25°.

19. The balloon catheter of claim, wherein the distal end of the balloon provides less crossing resistance when crossing a lesion due to increased taper length and/or shallower taper angle at the distal end of the balloon.

20. (canceled)

21. The balloon catheter of claim 1, wherein the first layer is an outer layer, wherein an outer surface of the outer layer of the multilayer inflatable balloon includes a hydrophilic coating for improved lubricity.

22. The balloon catheter of claim 1, wherein compliance of the multilayer inflatable balloon at an inflation pressure of from about 2 atm to about 5 atm is less than about 1% per atm.

23. (canceled)

24. (canceled)

25. (canceled)

26. (canceled)

27. (canceled)

28. (canceled)

29. (canceled)

30. A balloon catheter for use in a lithotripsy procedure, comprising:
an elongate catheter shaft;
an inflation lumen;
a multilayer inflatable balloon in fluid communication with the inflation lumen located toward, or at, a distal section of the elongate catheter shaft; and
at least one lithotripsy emitter within the multilayer inflatable balloon capable of generating an acoustic pressure wave within an inflation medium when the multilayer inflatable balloon is inflated;
wherein the multilayer inflatable balloon comprises:
a first layer and a second layer having a combined wall thickness;
the first layer being made of a first polymer material having a first maximum blow-up-ratio;
the second layer being made of a second polymer material having a second maximum blow-up-ratio greater than the first maximum blow-up-ratio; and
wherein the second layer is an inner layer relative to the first layer.

31. (canceled)

32. The balloon catheter of claim, wherein the balloon catheter is configured as a lithotripsy balloon catheter for use in heart valve lithotripsy or heart valve leaflet lithotripsy.

33. A method of manufacturing a balloon catheter, the method comprising:
providing a balloon catheter comprising:
an elongate catheter shaft and an inflation lumen;
a multilayer inflatable balloon in fluid communication with the inflation lumen located toward, or at, a distal section of the elongate catheter shaft; and
at least one lithotripsy emitter within the multilayer inflatable balloon capable of generating an acoustic pressure wave within an inflation medium when the multilayer inflatable balloon is inflated;
wherein the multilayer inflatable balloon comprises:
a first layer and a second layer having a combined wall thickness;
the first layer being made of a first polymer material;
the second layer being made of a second polymer material,
wherein the second layer is an inner layer relative to the first layer;
folding the multilayer layer balloon along one or more pleat lines, where the multilayer inflatable balloon is positioned about the elongate catheter shaft;
compressing the folded multilayer inflatable balloon using a balloon press;
removing the balloon press from about the multilayer inflatable balloon; and
positioning a balloon sheath over the compressed balloon to maintain a compressed geometry of the multilayer inflatable balloon.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. The balloon catheter of claim 1, wherein a second maximum blow-up-ratio of the second polymer is about 15 to about 40 percent greater than a first maximum blow-up-ratio of the first polymer.

52. (canceled)

53. (canceled)

54. (canceled)

55. The balloon catheter of claim 1, wherein the first layer has a first Shore D durometer hardness and the second layer has a second Shore D durometer hardness lower than the first Shore D durometer hardness.

56. (canceled)

57. (canceled)

58. (canceled)

59. (canceled)

60. (canceled)

61. The balloon catheter of claim 1, wherein the first polymer material and/or the second polymer material comprises at least one of a polyamide, a polyurethane, a polyester, or a polyether block amide.

62.

63. (canceled)

64. (canceled)

65. (canceled)

66. (canceled)

67. (canceled)
